(19) United States
(12) Patent Application Publication
(10) Pub. No.: US 2013/0071853 A1
(43) Pub. Date: Mar. 21, 2013

(54) METHODS FOR IDENTIFYING COMPOSITIONS THAT ALTER WILDTYPE EXPRESSION OF GENES AND PROTEINS IN A PLANT CELL

(71) Applicants: Jon Frederick Fobes, Lower Gwynedd, PA (US); Jeffrey L. Rosichan, Carmel, IN (US); Delkin Orlando Gonzalez, Indianapolis, IN (US)

(72) Inventors: Jon Frederick Fobes, Lower Gwynedd, PA (US); Jeffrey L. Rosichan, Carmel, IN (US); Delkin Orlando Gonzalez, Indianapolis, IN (US)

(73) Assignees: Dow Agrosciences LLC, Indianapolis, IN (US); Rohm and Haas Company, Philadelphia, PA (US)

(21) Appl. No.: 13/697,117

(22) PCT Filed: May 26, 2011
(86) PCT No.: PCT/US2011/038119

§ 371 Date: Dec. 7, 2012

Related U.S. Application Data
(60) Provisional application No. 61/349,577, filed on May 28, 2010.

(51) Int. Cl. C12Q 1/68

(52) U.S. Cl. CPC C12Q 1/68
USPC 435/6.13

(57) ABSTRACT

A method for identifying compounds that selectively alter wildtype gene expression of a plant cell includes comparing a first expression profile from a plant cell or tissue contacted with a first test compound, an additional expression profile from the same plant cell or tissue contacted with an additional test compound, and a combined compound expression profile from the same plant cell or tissue contacted with the first test compound and the additional test compound, wherein each plant cell or tissue was contacted for a time sufficient to produce an alteration in the expression of at least one gene or gene product in the plant's cells or tissues; and wherein each expression profile comprises the expression level or pattern of at least one gene or gene product of the plant cell or tissue; and identifying a significant, unexpected alteration in the level or pattern of expression between the genes or gene products of the combined compound profile and those of the additive alterations of the first profile and the additional profile.

### BACKGROUND

The search for desirable compositions and methods for treating plants, seeds, tissues and organs to regulate or modulate the physical characteristics of plants has involved identifying a target gene or protein responsible for the desired characteristic and then screening a variety of “test” compounds for the effect on the target gene/protein. The plant is generally contacted with each test compound and then grown under conventional circumstances to determine the effect of one or more test compounds as against a wildtype plant. Such screening techniques require considerable time in both identifying the target genes involved and in growing the plant to observe the effect of each test compound.

More recently, methods for identifying target plant genes or proteins have been suggested to facilitate compound screening. See, e.g., US Patent Application Publication No. US 2005/0221290 and US Patent Application Publication No. US2007/0265164.

There remains a need in the art for simpler and more rapid methods of identifying compounds that affect wildtype gene/protein expression in a plant cell.

### SUMMARY

The compositions and methods described herein provide for rapidly identifying combinations of test compounds that selectively alter wildtype gene expression or protein expression in a plant cell. The methods described permit the mechanistic determination of how compounds behave and interact prior to having to grow a plant to full physiological maturity in order to obtain such information. These methods provide a genetic evaluation of the effect of test compounds on the plant.

In one aspect, a method for identifying combinations of compounds that selectively alter wildtype gene expression of a plant cell is described. One such method includes comparing (a) a first expression profile from a plant cell or tissue contacted individually with a first test compound, (b) an additional expression profile from the same plant cell or tissue contacted individually with an additional test compound, and (c) a combined expression profile from the same plant cell or tissue contacted with a combination of the first test compound and the additional test compound. In other embodiments of the method, the additional compound comprises two or more additional compounds. Any background-subtracted, significant alteration in the level or pattern of expression of multiple genes or gene products in the combined compound profile (c) as compared to those of the first profile (a) and additional profile (b) identifies a combination of two or more compounds useful in affecting a growth characteristic of the plant. In one embodiment, the alteration of expression profiles is synergistic. In another embodiment, the alteration of expression profiles is antagonistic.

In any of the above-described methods, the comparing step is optionally performed by a computer processor or computer-programmed instrument that generates numerical or graphical data.

In another embodiment, a composition for affecting a plant growth characteristic is prepared by combining a first test compound (a) with an additional test compound (b) in a single composition or kit for simultaneous or concurrent application to a plant.

Other aspects and advantages of these methods and compositions are described further in the following detailed description.

### DESCRIPTION OF THE DRAWINGS

FIG. 1 is a graph showing the number and distribution of corn genes differentially expressed by application of compounds, 1-methylcyclopropene (MCP) vs. corn gene (UT), strobilurin (STB) vs. UT, or the combination of MCP and STB vs. UT. The intersecting circles demonstrate that when a corn plant is treated with MCP, the expression of 934 genes (Entity List 1) are changed significantly (≧2 fold) compared to untreated, with a subset of 362 genes changed uniquely in response to this condition. When the same plant is treated with the fungicide strobilurin, the expression of only 185 genes (Entity List 2) are significantly changed, with changes to 78 genes unique to this condition. However, when the combination of these two compounds is applied to the corn plant, 1128 total genes are changed (Entity List 3) in expression, with only 568 gene changes unique to the combination.

FIG. 2 is a graph showing the distribution of Arabidopsis genes differentially expressed by application of 1-methylcyclopropene (MCP) vs. untreated Arabidopsis gene (UT), strobilurin (STB) vs. UT, or the combination of MCP/STB vs. UT. The intersecting circles demonstrate that when an Arabidopsis plant is treated with MCP, the expression of 411 genes (Entity List 1) are statistically changed 5 fold) compared to untreated, with 202 unique gene expression changes. When the same plant is treated with the fungicide strobilurin, the expression of only 278 genes (Entity List 2) are statistically changed with 80 genes unique to this condition. When the combination of these two compounds is applied to the plant, however, only 93 genes (Entity List 3) are significantly changed in expression with only 44 unique gene changes.

### DETAILED DESCRIPTION

The compositions and methods described herein provide for rapid and efficient identification of combinations of compounds that impact a growth characteristic of a plant. The methods described herein are characterized by the absence of any need to identify a specific target gene(s) before performing the method. Similarly these methods do not require enhanced expression of any specified gene, but rather rely upon a pattern of alteration of expression of multiple genes/proteins. The pattern can include up-regulation and/or down-regulation of multiple genes in response to a combination of test compounds.

As used herein, the term “plant cell or tissue sample” refers to a single plant cell or culture of plant cells or whole or part of the plant. Representative cells or cell cultures or plant tissue may derive from a single specific cell type, a combination of cell types; a single specific tissue type; a combination of tissue types; a single specific plant organ; or a combination of plant organs. Such cells are selected from plant tissues and organs including, without limitation, vegetative tissues, e.g., roots, stems, or leaves, and reproductive tissues, such as fruits, ovules, embryos, endosperm, integument, seeds, seed coat, pollen, petal, sepal, pistils, flowers, anthers, or any embryonic tissue.

As used herein, the term “plants” are selected from dicotyledons or monocotyledons. Such plants include decorative, flowering plants as well as plants or plant parts for human or animal consumption. For example, a suitable plant contributing the cells used in these methods may be rice, maize, wheat, barley, sorghum, millet, switchgrass, miscanthus, grass, oats, tomato, potato, banana, kiwi fruit, avocado, melon, mango, cane, sugar beet, tobacco, papaya, peach, strawberry, raspberry, blackberry, blueberry, lettuce, cabbage, cauliflower, onion, broccoli, brussel sprout, cotton, canola, grape, soybean, oil seed rape, asparagus, beans, carrots, cucumbers, eggplant, melons, okra, parsnips, peanuts, peppers, pineapples, squash, sweet potatoes, rye, cantaloupes, peas, pumpkins, sunflowers, spinach, apples, cherries, cranberries, grapefruit, lemons, limes, nectarines, oranges, peaches, pears, tangelos, tangerines, lily, carnation, chrysanthemum, petunia, rose, geranium, violet, gladioli, orchid, lilac, crabapple, sweetgum, maple, poinsettia, locust, ash, poplar, linden tree and Arabidopsis.

Such plant cells are characterized by the expression of a distinctive set of nucleic acid molecules. At any one physical state of the plant, certain of these nucleic acid molecules, e.g., plant genes, or the products encoded thereby, are expressed more or less than others, thereby providing a pattern of expression of multiple genes or gene products. This pattern can be referred to as an expression “profile” or “fingerprint” that can identify the physiological state of the cells. Thus, the term “expression profile” refers to the pattern of expression of an identifiable set of molecules within the cell or cell culture. Representative molecule types that can be characterized for expression profiles include mRNA transcripts or cDNA derived therefrom; proteins; phosphoproteins; carbohydrates; lipids, metabolites; or any combination or permutation of mRNA transcripts, proteins, phosphoproteins, carbohydrates, lipids and metabolites. Such expression profiles may be obtained to represent a wild-type or background profile at any one physiological state or an altered profile observed in the cell when the plant is in a particular physical state, e.g., it has been exposed to a chemical agent or an environmental condition.

In one embodiment, the expression profile is a complete genome or complete set of encoded gene products of the plant cell. In another embodiment, each expression profile comprises a subset of genes or gene products encoded by the complete genome of the plant. In certain embodiments, the gene product is a plant protein. In still other embodiment, the profile tracks a corresponding change in a plant metabolite as a result of the alteration of one or more gene products in the profile.

In one embodiment of the methods described herein, the subset of genes or gene products forming a relevant expression profile comprises 3, 4, 5, 6, 7, 8, 9 or 10 or more genes or gene products. In another embodiment, the subset of genes or gene products forming the expression profile comprises 20, 30, 40, 50, 60, 70, 80, 90 or 100 or more genes or gene products. Still other expression profiles useful in the methods described herein are formed by 150, 200, 250, 300, 350, 400, 450 or 500 or more genes or gene products. Still other expression profiles useful in the methods described herein are formed by 750, 850, 1000, 1500, 2000 or more genes or gene products, including up to the entire genome of the plant cell. Depending on the number of genes obtained showing differential expression, a cutoff of ≧2 fold change or ≧5 fold change or is most typically selected to focus the analysis on the most meaningful subset. To form a useful expression profile for the methods described herein, the expression profiles of the plant cell evaluated must be significantly altered, both from the background expression profile of the same plant cell samples, as well as from the expression profiles of the first test compound and the additional expression profiles.

As used herein, the term “physical state” of the plant includes without limitation the conditions of increased ethylene sensitivity, decreased ethylene sensitivity, enhanced ripening, increased resistance to stress, heat, population density or salinity, decreased resistance to stress, heat, population density or salinity, increased pathogen resistance, decreased pathogen resistance, increased flowering, increased nitrogen efficiency, increased herbicidal resistance, increased photosynthetic activity and increased yield.

As used herein the term “test compound” or “additional compound” means a chemical compound that is being tested for its impact on a plant's expression profile when exposed to a plant, plant cell or cell culture. Such compounds can include compounds with known or unknown effect on a plant when employed individually. Such compounds can include known plant disease or regulatory agents, or can be unknown compounds obtained using any of the numerous approaches in combinatorial library methods known in the art. The test compounds may be presented in a suitable carrier, diluent or solution, and may be applied by immersing, spraying, powdering, drenching, dripping, or irrigating the plant or plant cells.

To generate the expression profiles for use in the methods described herein, each plant or plant cell, or tissue is from the same plant and is substantially identical in composition, e.g., number and density of cells, cell media, culture conditions, plant growth conditions, etc. Each plant, plant cell or culture is contacted for a time sufficient to produce an alteration in the expression of at least three genes or gene products in the plant's cells or tissues with one of a first test compound; or an additional test compound, or a combination of the first test compound and the additional test compound. “A time sufficient” is generally defined as at least 5, 10, 15, 25, 30, 45, or 60 minutes, or at least 1, 2, 3, 4, 5, 6 up to 12 hours or 24 hours. The compounds of the combination may be applied to the plant or plant cells simultaneously or consecutively. Further in certain embodiments, the additional test compound is more than a single compound and may itself be a mixture. In certain embodiments, the first test compound is a compound with a known effect upon the plant cell and the additional compound is a compound with an unknown effect upon the cell, or vice versa. In other embodiments, both the first test compound and the additional test compound have unknown individual effects upon the cell. Similarly the amounts of the test compounds needed for use in this method are considerably smaller than those required for experimentation in the field or in growing the plant in the laboratory. Suitable amounts of the compositions for use in cells or cell cultures will, of course, depend upon the identify of the compounds themselves, but can include at least 0.2, 0.5, 0.8 or 1.0 or more micrograms per milligram cells in the culture or the same amount in mL of diluent for application to the plant or plant tissue. In another embodiment, the amounts of test compound comprises at least 2, 5, 8 or 10 or more micrograms per milligram cells in the culture or the same amount in mL of diluent for application to the plant or plant tissue. In another embodiment, the amounts of test compound comprise at least 2, 5, 8 or 10 or more milligrams per milligram cells in culture or the same amount in mL of diluent for application to the plant or plant tissue.

After the plant cell, tissue or plant is contacted, tissue samples are collected within 2, 5, 10, 20, 30, 40, 50 or 60 minutes, or 2, 4, 6, 8, 10, 12, 14, 16, 18, 20, 24 hours following contact with the compound. In some embodiments, tissue may be sampled 2, 4, 6, 8, 10, 14, 16, 18, 20, 22, 24, 26, 28, 30 days following contacting with the compounds. In yet other embodiments, sampling may be done monthly up to 6 months following contacting the plant cell, tissue or plant with the compounds in order to access longer term down stream effects of the compounds.

The method further includes a step of generating an expression profile from the plant cell or tissue contacted with the first test compound individually, an expression profile from the plant cell or tissue contacted with the additional compound(s) individually; and a combined compound expression profile from the plant cell or tissue contacted with the combination of the test compound and additional compound.

The generation and characterization of the expression profiles may be conventionally obtained by one or more of the following methods: application of appropriately prepared samples to polynucleic acid microarrays of plant genomes or the products encoded thereby. Examples of such microarrays have been described by Affymetrix, Inc. or numerous other manufacturers of microarrays. The generation or use of microarrays may be followed by mRNA expression pattern detection; 2-dimensional gel electrophoresis of appropriately prepared samples to derive a pattern of protein expression; application of samples to arrays of antibodies to derive a profile of protein expression; application of samples to arrays of polynucleotides that differentially bind to specific peptides to derive a pattern of protein expression. Similarly, analysis of appropriately prepared samples by mass spectrometry may be employed to derive mRNA or protein expression patterns. The analysis may be conducted on appropriately prepared samples by means of application to bead-based mRNA and protein expression analytic methods, such as that described by Lynx Therapeutics, Inc., Illumina, Inc., or Luminex, Inc., to derive mRNA or protein expression patterns. The techniques described in the examples below may also be employed for these purposes. For use in the methods described herein, any method other than the above that characterizes a distinctive profile of expression of multiple molecular components of samples may be employed. See, for example, the techniques for generation of expression profiles described in US2007/0265164; US2005/0221290; or in the pharmaceutical patent U.S. Pat. No. 7,332,272, among other known descriptions of expression profile generate extant in the art.

In certain embodiments, the expression profiles are generated by extracting, enzymatically amplifying and labeling mRNA of a plant with a detectable signal-generating agent This labeled nucleic acid molecule is then exposed to a microarray upon which have been discretely spotted DNA sequences complimentary to many or all of the possible mRNA species expressed by the plant cell. The labeled nucleic acid molecules that are expressed by the plant cell hybridize differentially to the discrete spots, conferring a detectable signal proportional to the concentration of each molecule in the sample. The intensity of the signal of each of the spots is detected and quantified rapidly using established technology, e.g., a microarray reader. The expression levels of those same mRNA transcripts or the entire genome for the various plant cells (e.g., wildtype plant cell, first compound treated plant cell, second compound treated plant cell, and plant cell treated with both test compounds) are determined. When each expression profile is generated and before the results are analyzed by suitable clustering or other techniques, background expression levels of the genes/gene products forming the expression profiles are subtracted. The background expression levels are obtained from a profile generated from the same type of plant, plant cells/cultures, which have been exposed to the same culture and environmental conditions as the “test” plant cells or cultures, but have not been contacted with the first compound or any additional compound.

Thereafter the individually treated profiles are compared to the combined profile to identify mRNA transcripts with different expression levels and patterns between the differently treated plant cells. So-called “additive expression profiles” are the anticipated calculated results of a combination of each individually treated expression profiles.

The method described herein can be performed in a high-throughput analysis using plant cell cultures rather than whole plants. For example, thousands of separate cultures of plant cells are established in 96-well or more culture plates, with each well treated with a different test compound. The RNA is treated and extracted as described above. Each sample is then exposed to a separate microarray of the wildtype plant cell. Each of the microarrays is read by a microarray reader or multiple microarray readers to derive the effect of each test compound on the plant cell expression profile. Expected additive profiles are calculated and the expected results compared to the actual combined expression profile using rigorous statistical analysis.

Expression profiling may also utilize the simultaneous detection of the rates of transcription of many genes. The rates are obtained by rapid sequential array measurements of cells, tissue or the plant undergoing some perturbation. The array of rates, and rates of change of the rates of expression levels can be considered independently or in combination with absolute expression levels to obtain a more informative expression profile. Thus, one skilled in the art could apply simultaneous detection of the rates of expression of many biological molecules, such as mRNA transcripts, to the methods described herein.

The methods described herein further comprise identifying a significant alteration in the level or pattern of expression between the genes or gene products of the combined compound expression profile and those forming the expression profiles of the cells/culture contacted individually with the first test compound and the additional compound by comparing the respective expression profiles. This comparing step may be performed by visual inspection of the profiles or may desirably be performed by a computer processor or computer-programmed instrument that generates numerical or graphical data. Each alteration of expression of a gene or gene product forming each expression profile is expressed as a mean or average, a numerical mean or range of numerical means, a numerical pattern, or a graphical pattern.

In one embodiment, the alteration is an upregulation of the same selected gene(s) or gene product(s) in the combined compound profile compared with the additive expression of the same gene(s) or gene product(s) in the first test compound profile and the additional test compound profile. In another embodiment, the alteration in the combined profile is a downregulation of the same selected gene(s) or gene product(s) in the combined compound profile compared with the additive expression of the same gene(s) or gene product(s) in the first test compound profile and the additional test compound profile.

In another embodiment, the alteration is a change in expression of one or more selected gene(s) or gene product(s) in the combined compound profile which selected gene or gene product was unaltered from wildtype levels in a profile generated from the additive alterations of the first profile and the additional profile. For example, the gene(s) or gene product(s) altered from wildtype levels of expression in the combined compound expression profile are different gene(s) or gene product(s) that were altered in the first test compound profile, the additional test compound profile, or a additive profile predicted by the individual profiles. Alternatively, the same gene(s) or gene product(s) form the compared expression profiles, but the alteration involves an upregulation of the same selected genes or gene products in the combined compound profile that were unaffected or down-regulated in the individual or predicted additive expression profiles of the first profile and the additional profile. Still alternatively, a significant alteration involves a downregulation of the same selected genes or gene products in the combined compound profile that were unaffected or up-regulated in the individual or predicted additive expression profiles of the first profile and the additional profile. Still another significant alteration between the combined expression profile and the individual expression profiles (or predicted additive profile anticipated therefrom) involves a change in the identity of the individual genes or gene products forming the combined profiles vs those forming the individual or predicted additive profiles formed by the first test compound profile and the additional test compound profile. For example, some genes/gene products that were unaltered from wildtype levels in a profile generated from the additive alterations of the first profile and the additional profile were up- or down-regulated in the combined profile and other genes/gene products that were up- or down-regulated from wildtype levels in a profile generated from the additive alterations of the first profile and the additional profile were not altered from wildtype levels of expression in the combined profile.

In one embodiment, the identification of a significant alteration in the combined compound expression profile correlates with a selected effect on the plant cells, e.g., a different effect than that caused by either the test compound or additional compound alone or a different, greater or lesser effect than the predicted additive effect of the two compounds. In one embodiment, the altered effect is a significant enhancement or diminution of the effect caused by one of the test compound and diminution of the effect caused by the other. In one embodiment, the altered effect is a significant increase in the effect that would be anticipated to be produced by the additive use of the test compounds and additional compound based upon their expression profiles. In still another embodiment, the combined expression profile causes an effect that is different from the effect caused by either of the individual effects alone or the anticipated additive effects of the test and additional compounds.

In another embodiment, the combination of compounds has a synergistic effect that is a greater alteration in expression patterns or levels of the gene(s) or gene product(s) of the plant in the combined compound profile than that predicted by the additive alterations in patterns or levels of the first and additional expression profiles. In certain embodiments, the alteration in the pattern or levels of expression in the combined expression profile is an at least 2-fold alteration in either up-regulated or down-regulated expression of the genes or gene products forming the pattern of the additive alterations of the first and additional profiles. In other embodiments, the alteration is at least 3, 4, 5, 6, 7, 8, 9, 10 or greater than 20-fold that expected by predicting the additive effect (up-regulation or down-regulation) of the two compounds based on their expression profiles.

In still a further embodiment, the combination of compounds has an antagonistic effect that is a reduction in the magnitude of the alteration in expression pattern or level of genes or gene products in the combined compound profile than that produced by the pattern or level of expression of the additive alterations of the first and additional profiles. This type of result indicates that the two compounds have antagonistic effects on the plant cells.

Thus, by applying the method of this invention, and comparing the expression profiles generated by combined treatment of plant cells with different compounds to the individual expression profiles of the two or more compounds, or their calculated additive expression profile results, this method provides a rapid and efficient screening of combinations of compounds for use in treating a variety of plant diseases or conditions, e.g., the effects of stress, to delay ripening, etc, without the necessity to grow the plants in the compounds.

These methods and the results obtained thereby further permit the formulation of compositions or kits for treating plant diseases or manipulating the physical states of plants. Each composition or kit comprises an effective synergistic amount of the at least two compounds identified by the methods described herein. Those compounds can be physically admixed into a single formulation for application to the plant, or may be present in a kit in separate containers for simultaneous or consecutive application to the plant in the field.

The following examples illustrate certain embodiments of the above-discussed method generated by using a first test compound, the plant growth regulator, 1-methylcyclopropene (1-MCP), and as the additional test compound, the COMPASS™ fungicide trifloxystrobin in a 50% w/w water-dispersible granule formulation (Bayer Environmental Science, North Carolina). Strobilurin has been observed to have some effects on plant health, in addition to its benefits as a fungicide. While both of these compounds are known and also are claimed to be synergistic, they are employed to demonstrate the performance of the claimed methods. These examples do not limit the disclosure of the claims and specification.

### EXAMPLE 1

### Growth and Treatment of Corn Plants

Hybrid corn seed (one plant per pot) was planted in 1 quart pots filled with FAFARD 3B™ potting mix (Conrad Fafard, Inc., Agawam, Mass.). Corn was grown in a greenhouse with a 12 hr light cycle and watered every 24 hours or as needed to maintain stress free conditions. When the corn plants reached the V10 growth stage, the pots were arranged into a randomized complete block design with 4 plants with 3 replications per treatment. Corn plants were treated using a standard CO₂ sprayer with 20 gal/acre carrier volume.

Treatments were:
Control: Oil/Adjuvant check using 0.035% v/v the organosilicone surfactant SILWET L-77 (Helena Chemical Co., Collierville, Tenn.)
Compound 1: A17492F (MCP) 25 g active ingredient/hectare (ai/ha)+0.035% SILWET surfactant
Compound 2: COMPASS fungicide 1x+0.035% SILWET surfactant
Combination (1 and 2): 1 MCP 25 g/ha+COMPASS fungicide 1x

Two hours after the spray application, upper leaves that had received the spray were sampled. For each plant, five, 2 inch pieces of leaves were taken, cut into small pieces and combined into a 50 ml polypropylene tube. Samples were immediately frozen on dry ice and sent on dry ice overnight to the laboratory for processing.

### EXAMPLE 2

### Corn RNA Preparation and Hybridization

A. Experimental Design

A single-color Agilent global gene expression profiling design was used to define differentially expressed genes for treated in reference to untreated maize leaf tissues. Three technical array replicates (Agilent Technologies Inc., Santa Clara, Calif.) were hybridized to compare different transcript abundance between leaf tissues treated with the COMPASS fungicide and/or 1-MCP in reference to untreated leaf tissues.

A Dow AgroSciences custom generated 60-mer comprehensive transcriptome-wide Zea maize oligonucleotide array was used to carry out microarray hybridizations. This array represents more than 58,000 different maize transcripts (Agilent Technologies Inc., Santa Clara, Calif.) obtained from public data sources. Microarray chips were printed using an Agilent format 2×105K, including 10 copies of 100 probes, 2 copies of 44,196 probes 1 copy of 14,355 probes in addition to 1,325 Agilent spike-in controls. The 60-mer unique and specific oligonucleotides were synthesized in-situ using the Sure-Print technology from the manufacturer.

B. RNA Isolation and Purification

Samples of 30-50 mg of frozen leaf tissues were resuspended in 450 μL of extraction buffer RLT from the RNeasy™ Kit for RNA extraction (Qiagen, Valencia, Calif.). Tissues were then ground to a fine powder by adding one 3 mm grinding bead to each frozen sample and grinding for 3 minutes using a GenoGrinder instrument at a rate of 300, 1×. Total RNA was purified following the manufacturer's instructions. Purified total RNA was then quantified and quality controlled using a NanoDrop™ spectrophotometer and visualized by standard 1% Agarose gel electrophoresis.

C. cRNA Labeling

For labeling, a total of 1.0 μg of purified RNA from treated and untreated tissues was reverse transcribed, amplified and labeled with Cy3-CTP following the Agilent (Santa Clara, Calif.) one-color microarray-based gene expression QuickAmp™ labeling protocol. Since Dow AgroSciences-generated Zea maize AGILENT™ microarrays for gene expression (P/N G4503A, Design ID 022232) contain internal spike-in controls a one-color RNA spike-in kit (Agilent, Santa Clara, Calif.) was also labeled according to manufacturer's instructions. Samples were reverse transcribed using MMLV Reverse Transcriptase and amplified using a T7 RNA Polymerase. After amplification cRNA was purified using Qiagen's RNeasy mini spin columns and quantified using a NanoDrop spectrophotometer. Specific activity for Cy3 was determined by the following formula:

(Concentration of Cy3)/(Concentration of cRNA)* 1000=pmol of Cy3 per μg of cRNA.

Samples for hybridization were normalized to 825 ng with a specific activity of >8.0 pmol of Cy3 per μg of cRNA.

D. Hybridization

Oligo gene expression arrays were hybridized using the Agilent Technologies (Santa Clara, Calif.) Gene Expression Hybridization kit, Wash Buffer kit, Stabilization and Drying Solution following the manufacturer's instructions. Hybridizations were carried out on a fully automated TECAN HS4800 PRO™ (TECAN U.S., Research Triangle Park, N.C.). The hybridization mixture was injected at 65° C. and incubated with agitation for 17 hrs after following a slide pre-hybridization step at 65° C. for 30 seconds. Slides were then washed at 37° C. for 1 minute using the Agilent GE Wash #1 followed by a second wash at 30° C. with Agilent GE Wash #2 for 1 minute and a final drying step using Nitrogen gas for 2 minutes and 30 seconds at 30° C. Slides were scanned immediately to minimize impact of environmental oxidants on signal intensities.

E. Scanning, Feature Extraction and QC Metrics

Arrays were scanned using an Agilent G2565CA microarray laser scanner with SureScan™ high resolution technology (Agilent Technologies, Santa Clara, Calif.). The protocol for scanning each array defines parameters for dye channel, scan region and resolution, TIFF file dynamic range, PMT gain and the setting for the final image outcome. Once the array has been scanned a feature extraction protocol is followed with parameters defined for placing and optimizing the grid fit, finding the spots, flagging outliers, computing background bias, error and ratios, and calculating quality control metrics.

After scanning and feature extraction protocols are completed, a TIFF file containing the Cy3 image is generated along with a quality control metrics report and a final file (TXT) containing all the raw data. The image files (TIFF) were used to examine general quality of the slides, presence of spike-in controls in the right positions (four corners) and intensities as well as to confirm that the hybridization, washing, scanning and feature extraction processes were successful. The quality control (QC) report provided values of coefficient of variation and was used to measure dispersion of data based on positive and negative (prokaryotic genes and artificial sequences) spike-in controls provided and designed by Agilent Technologies (Santa Clara, Calif.). This report was used to determine data distribution, uniformity, background, reproducibility, sensitivity and general quality of data. The TXT file containing all the raw data was uploaded into GeneSpring™ program (Agilent, Santa Clara, Calif.) for analysis.

F. Data Uploading, Normalization, Filtering and Statistical Analysis

After scanning and feature extraction, raw data files were uploaded into GeneSpring™ GX version 10.0.2 (Agilent Technologies, Santa Clara, Calif.) and a project was created defining each array data file as a sample and assigning the appropriate parameter values. Samples with the same parameter values were treated as replicates. Interpretations were created to specify how the samples were grouped into experimental conditions and were used to visualize and analyze data. Quality control on samples based on spike-in controls was performed to ensure quality of data before starting analysis. A report was generated.

Data was normalized using a global percentile shift normalization method to minimize systematic non-biological differences and standardize arrays for cross comparisons. Data was then filtered based on expression levels between the 20th and 100th percentile in the normalized data and restricted to have a significant value within cut-off in every sample under study. Another set of filtering was done by selecting entities that were flagged as Present in every single sample under study and eliminating entities flagged as Marginal or Absent.

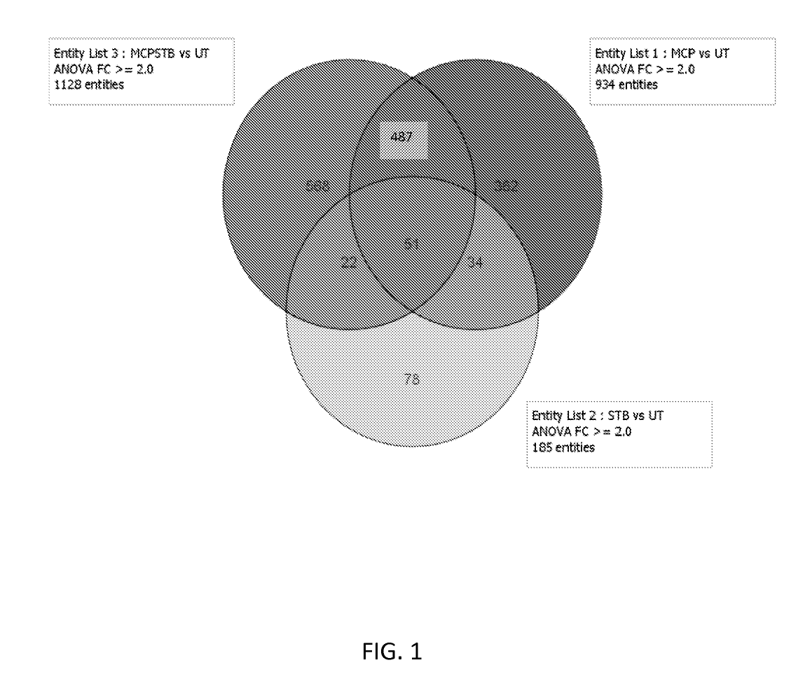

Using this final list of entities (genes represented in the array) as input a statistical analysis using a One-way ANOVA with a corrected p-value P≦0.05 and a Benjamin-Hochberg multiple testing correction FDR of 0.05. A final list of significant entities was then filtered by a specific fold change and used for data interpretation. FIG. 1 shows the results of Examples 3, 4 and 5 graphically.

### EXAMPLE 3

### Corn Gene Expression Induced by 1-Methylcyclopropene

Treatment of corn with 1-methylcyclopropene (1-MCP) resulted in the differential expression (≧2 fold change) of 934 genes on the microarray. From these, a subset of 362 genes unique to the 1-MCP treatment was further analyzed. For this analysis we focused on annotated genes and looked for changes/trends within related functional groups of genes. The expression and putative function of these genes is shown in Table 1.

Analysis of the specific genes in Table 1 suggests that the expression of 9 out of 10 genes involved in translation whose expression changed in this particular experiment was induced with only one being repressed. As global translation is typically one of the first processes to be repressed in response to abiotic and biotic stresses, the expression of components of the general translational machinery can serve as an indicator of the general level of stress in the plant. These data suggest that the plants were not stressed and may have experienced a reduced level of stress. Genes such as glutathione transferase or thioredoxin related proteins often are induced during conditions of stress and the reduction in expression of these genes observed is consistent with a reduced level of stress. In the third category, the expression of genes encoding proteins whose expression is induced by specific stresses (e.g., universal stress protein), decreased indicating a reduction in stress, whereas the expression of beta-expansin, involved in cell expansion, increased, consistent with a reduction in stress. The expression of viviparous 14, which is involved in ABA production, increased and as ethylene and ABA often oppose each other, this might indicate a reduction in ethylene signaling by the 1-MCP treatment. Taken overall, the 1-MCP treatment appears to have resulted in a reduction in stress-related gene expression with an increase in the expression of a set of genes reflecting more active metabolism. Table 1, following the last Example 10, lists informative corn genes differentially expressed in response to 1-MCP alone.

### EXAMPLE 4

### Corn Gene Expression Induced by Compass Fungicide

Treatment of corn with the COMPASS strobilurin fungicide resulted in the differential expression (≧2 fold change) of 185 genes on the microarray. Of these, there was a subset of 78 genes that were unique to the COMPASS treatment. A low number of differentially expressed genes was observed in this treatment in comparison to the treatment with 1-MCP. This may be due to the COMPASS strobilurin fungicide requiring more than 2 hours to produce significant changes at the level of gene expression following treatment.

### EXAMPLE 5

### Corn Gene Expression Induced by the Combination of 1-MCP and Compass Fungicide

Treatment of corn with the combination of 1-methylcyclopropene and the strobilurin fungicide, Compass resulted in the differential expression (≧2 fold change) of 1128 genes on the microarray. From these, a subset of 568 genes unique to the combined treatment was further analyzed. For this analysis we focused on annotated genes and looked for changes/trends within related functional groups of gene. FIG. 1 shows the graphical results of the comparison of conditions.

The expression and putative function of these genes is shown in Table 2. Analysis of these genes suggests that the expression of 3 out of 7 genes involved in translation whose expression changed in this particular experiment was induced with four being repressed. Unlike what was observed for treatment with 1-MCP alone, the combination treatment does not reveal any consistent trend with regard to the status of global translation. However, a decrease in the expression of genes such as glutathione transferase or ascorbate peroxidase was observed consistent with a reduced level of stress. 4 out of 5 of these genes whose expression changed in this particular experiment decreased with only one increasing. In the third category, the expression of 5 out of 7 genes encoding proteins whose expression is induced by specific stresses (e.g., temperature-induced), decreased indicating a reduction in stress, with only two increasing. Two genes involved in defense responses also decreased, suggesting a reduction in stress. The expression of herbicide safener binding protein, involved in binding safeners that protect maize against injury from some herbicides (e.g., chloroacetanilide and thiocarbamate) and induce production of glutathione or increase expression of glutathione transferase as a protective mechanism, increased. This same protein increases in the strobilurin only treatment, suggesting that this may be a gene which is induced in response to the strobilurin specifically. It may serve as a potential marker gene for strobilurin treatment/response. In another category, the expression of carbonic anhydrase, involved in converting CO₂ to bicarbonate, which assists in raising the concentration of CO₂ in the chloroplast for photosynthesis, increased. However, expression of this same gene decreased in the 1-MCP treatment. Also, the expression of three other genes involved in the Calvin cycle decreased in the 568 unique gene subset from the combination treatment, suggesting a possible decrease in photosynthesis.

In a further category, the expression of one histone deacetylase, which is an enzyme often involved in transcriptional induction, increased whereas the expression of another histone deacetylase decreased. A possible explanation is that different family members may work differentially under the same conditions. The expression of allene oxide synthase, involved in jasmonate biosynthesis, which is generally considered a stress-related hormone, decreased, suggesting a possible reduction in the stress level. Therefore, some changes indicate a possible reduction in stress whereas others may not. Taken together, these results suggest that the two compounds act antagonistically.

### EXAMPLE 6

### Growth and Treatment of Arabidopsis Plants

Thirteen Arabidopsis seeds (variety Columbia) were planted in 6 inch pots filled with sandy clay medium. After emergence, pots were thinned to 10 plants per pot. Plants were grown in a greenhouse with a 12 hr light cycle and watered every 24 hours or as needed to maintain stress free conditions. When the Arabidopsis plants reached the vegetative (>BBCH 11<BBCH 30) stage (approx. 2-3 wks from planting), the pots were arranged into a randomized complete block design with 10 plants with 3 replications per treatment. The plants were treated using a standard CO₂ sprayer with 20 gal/A carrier volume.

Treatments were:

Control: Oil/Adjuvant check (0.035% v/v SILWET adjuvant L-77)

Compound 1: A17492F (MCP) 25 g ai /ha+0.035% SILWET adjuvant

Compound 2: COMPASS fungicide 1x+0.035% SILWET adjuvant

Combination: 1 MCP 25 g/ha+COMPASS fungicide 1x

Two hours after the spray application, leaves that had received the spray were sampled. For each treatment, two leaves were taken from each of the 10 plants and combined into a 50 ml polypropylene tube. Samples were immediately frozen on dry ice and sent on dry ice overnight to the laboratory for processing.

### EXAMPLE 7

### Arabidopsis RNA Preparation and Hybridization

A. Experimental Design

A single-color Agilent global gene expression profiling design was used to define differentially expressed genes for treated in reference to untreated Arabidopsis leaf tissues. Three technical array replicates (Agilent Technologies Inc., Santa Clara, Calif.) were hybridized to compare different transcript abundance between leaf tissues treated with Strobilurin and/or 1-MCP in reference to untreated leaf tissues. Arabidopsis slides, Product #G2519F, Design ID 021169 were obtained from Agilent Technologies.

B. RNA Isolation and Purification

Samples of 30-50 mg of frozen leaf tissues were resuspended in 450 μL of extraction buffer RLT from the RNeasy Kit for RNA extraction (Qiagen, Valencia, Calif.). Tissues were then ground to a fine powder by adding one 3 mm grinding bead to each frozen sample and grinding for 3 minutes using a GenoGrinder at a rate of 300, 1×. Total RNA was purified following the manufacturer's instructions. Purified total RNA was then quantified and quality controlled using a NanoDrop spectrophotometer and visualized by standard 1% Agarose gel electrophoresis.

C. cRNA Labeling

For labeling, a total of 1.0 μg of purified RNA from treated and untreated tissues was reverse transcribed, amplified and labeled with Cy3-CTP following the Agilent (Santa Clara, Calif.) one-color microarray-based gene expression QuickAmp labeling protocol. Since Agilent Arabidopsis microarrays for gene expression (Product #G2519F, Design ID 021169) contain internal spike-in controls a one-color RNA spike-in kit (Agilent, Santa Clara, Calif.) was also labeled according to manufacturer's instructions. Samples were reverse transcribed using MMLV Reverse Transcriptase and amplified using a T7 RNA Polymerase. After amplification cRNA was purified using Qiagen's RNeasy mini spin columns and quantified using a NanoDrop spectrophotometer. Specific activity for Cy3 was determined by the following formula: (Concentration of Cy3)/(Concentration of cRNA)*1000=pmol of Cy3 per μg of cRNA. Samples for hybridization were normalized to 825 ng with a specific activity of >8.0 pmol of Cy3 per μg of cRNA.

D. Hybridization

Oligo gene expression arrays were hybridized using the Agilent Technologies (Santa Clara, Calif.) Gene Expression Hybridization kit, Wash Buffer kit, Stabilization and Drying Solution following the manufacturer's instructions. Hybridizations were carried out on a fully automated TECAN HS4800 PRO (TECAN U.S., Research Triangle Park, N.C.) hybridization instrument. The hybridization mixture was injected at 65° C. and incubated with agitation for 17 hrs after following a slide pre-hybridization step at 65° C. for 30 seconds. Slides were then washed at 37° C. for 1 minute using the Agilent GE Wash #1 followed by a second wash at 30° C. with Agilent GE Wash #2 for 1 minute and a final drying step using Nitrogen gas for 2 minutes and 30 seconds at 30° C. Slides were scanned immediately to minimize impact of environmental oxidants on signal intensities.

E. Scanning, Feature Extraction and QC Metrics

Arrays were scanned using an Agilent G2565CA microarray laser scanner with SureScan™ high resolution technology (Agilent Technologies, Santa Clara, Calif.). The protocol for scanning each array defines parameters for dye channel, scan region and resolution, TIFF file dynamic range, PMT gain and the setting for the final image outcome. Once the array has been scanned a feature extraction protocol is followed with parameters defined for placing and optimizing the grid fit, finding the spots, flagging outliers, computing background bias, error and ratios, and calculating quality control metrics. After scanning and feature extraction protocols are completed, a TIFF file containing the Cy3 image is generated along with a quality control metrics report and a final file (TXT) containing all the raw data. The image files (TIFF) were used to examine general quality of the slides, presence of spike-in controls in the right positions (four corners) and intensities as well as to confirm that the hybridization, washing, scanning and feature extraction processes were successful. The quality control (QC) report provided values of coefficient of variation and was used to measure dispersion of data based on positive and negative (prokaryotic genes and artificial sequences) spike-in controls provided and designed by Agilent Technologies (Santa Clara, Calif.). This report was used to determine data distribution, uniformity, background, reproducibility, sensitivity and general quality of data. The TXT file containing all the raw data was uploaded into GeneSpring (Agilent, Santa Clara, Calif.) for analysis.

F. Data Uploading, Normalization, Filtering and Statistical Analysis

After scanning and feature extraction, raw data files were uploaded into GeneSpring GX version 10.0.2 (Agilent Technologies, Santa Clara, Calif.) and a project was created defining each array data file as a sample and assigning the appropriate parameter values. Samples with the same parameter values were treated as replicates. Interpretations were created to specify how the samples were grouped into experimental conditions and were used to visualize and analyze data. Quality control on samples based on spike-in controls was performed to ensure quality of data before starting analysis. A report was generated.

Data was normalized using a global percentile shift normalization method to minimize systematic non-biological differences and standardize arrays for cross comparisons. Data was then filtered based on expression levels between the 20th and 100th percentile in the normalized data and restricted to have a significant value within cut-off in every sample under study. Another set of filtering was done by selecting entities that were flagged as Present in every single sample under study and eliminating entities flagged as Marginal or Absent.

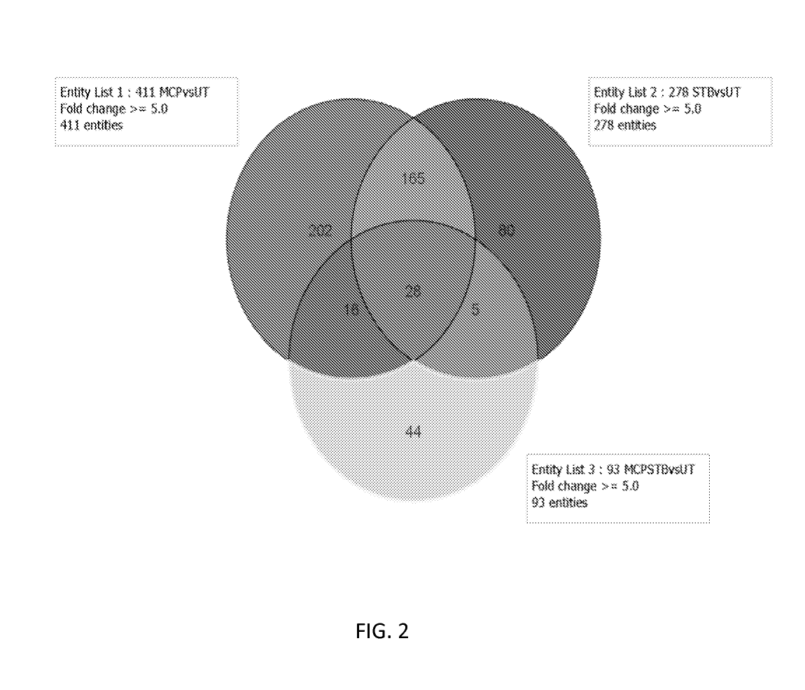

Using this final list of entities (genes represented in the array) as input a statistical analysis using a One-way ANOVA with a corrected p-value P≦0.05 and a Benjamin-Hochberg multiple testing correction FDR of 0.05. A final list of significant entities was then filtered by a specific fold change and used for data interpretation. FIG. 2 shows graphically, the comparison of genes significantly expressed by each condition of Examples 8-10, and the numbers of unique genes expressed by any of these conditions.

### EXAMPLE 8

### Gene Expression Induced by 1-Methylcyclopropene in Arabidopsis

Treatment of Arabidopsis with 1-methylcyclopropene (1-MCP) resulted in the differential expression (≧5 fold change) of 411 genes on the microarray. From these, a subset of 202 genes unique to the 1-MCP treatment was further analyzed. For this analysis we focused on annotated genes and looked for changes/trends within related functional groups of genes. The expression and putative function of these genes is shown in Table 3. The 1-MCP treatment as reflected in the unique MCP gene set appears to have resulted in a reduction in stress-related gene expression with a decrease in the expression of a set of genes known to ethylene-inducible, e.g., PDF1.2; chitinase, ACS2; SAG13. It was noted that α-chitinase is ethylene-inducible although it is unknown whether the chitinase genes represented in the microarray chips used are similarly regulated. Also, a decrease in the expression of a set of genes regulated by ABA in which ethylene-regulation often is involved suggests they may actually represent a true reduction in expression. Genes such as glutathione transferase or thioredoxin related proteins often are induced during conditions of stress and the reduction in expression of these genes observed is consistent with a reduced level of stress. Moreover, the expression of genes encoding proteins whose expression is induced by specific stresses (e.g., universal stress protein), decreased indicating a reduction in stress. This was also observed in the corn microarray experiment. An increase in the expression of ACS5 which is inhibited by wounding also is consistent with a reduction in stress. The expression of allene-oxide cyclase (AOC3) which catalyzes the first step in the biosynthesis of jasmonic acid, decreased, suggesting a possible reduction in the stress level. Similarly the expression of allene oxide synthase, involved in jasmonate biosynthesis which is generally considered a stress-related hormone, decreased in the corn microarray experiment. This suggests a possible reduction in the stress level. Ethylene induces the expression of allene oxide synthase. The expression of an F-box family protein decreased and some F-box proteins are down regulated by ethylene such as those regulating EIN3. The expression of several WRKY transcription factors decreased, some WRKY transcription factors are known to be induced, others repressed by ethylene. The expression of two AP2 domain-containing transcription factor family proteins decreased. Ethylene response factors (ERR), several of which are induced by ethylene, are members of the ethylene response factor/apetala2 family. The expression of other genes, i.e., methionine sulfoxide reductase domain-containing protein, S-adenosylmethionine-dependent methyltransferase/methyltransferase, MAPKKK19; ATP binding/kinase/protein kinase/protein serine/threonine kinase, protein kinase family protein; alternative oxidase; peroxidase; and MYB proteins, show a pattern but it is not known whether they are regulated by ethylene. Table 3, following the last Example 10, lists informative Arabidopsis genes differentially expressed in response to 1-MCP alone. The data demonstrate that taken overall, the 1-MCP treatment resulted in a reduction in stress-related gene expression.

### EXAMPLE 9

### Gene Expression Induced by COMPASS Fungicide in Arabidopsis

Treatment of Arabidopsis with the COMPASS strobilurin fungicide resulted in the differential expression (≧5 fold change) of 278 genes on the microarray. Of these, there was a subset of 80 genes that were unique to the COMPASS treatment. Annotated genes that can be grouped to provide insight into the effect of COMPASS treatment are shown in Table 4. Analysis of the 80 unique COMPASS alone treatment genes reveals some patterns in gene expression, including a decrease in Vegetative Storage Protein; Jasmonate-Zim-Domain Protein; Nitrile Specifier Protein; α-Amylase; O-methyltransferase family 2 protein; transferase family protein; JR1 which is a wound-responsive gene induced by JA; nucleoside phosphatase family protein; palmitoyl protein thioesterase family protein; cysteine proteinase; NAI2. However, it is not known whether they are regulated by ethylene. Of the remaining annotated genes, no groups of genes affecting a common pathway were observed and therefore no trends can be determined, consequently, conclusions based on these would be highly speculative. We observed a low number of differentially expressed genes in this treatment. The COMPASS strobilurin fungicide may require more than 2 hours to produce significant changes at the level of gene expression following treatment.

The fact that there are 165 common genes following treatment with either 1-MCP or COMPASS alone suggests that there are more genes in common between the two treatments than are unique to strobilurin treatment alone, suggesting that strobilurin treatment may alter a subset of the same genes that 1-MCP treatment does. Informative genes that are common to each treatment are shown in Table 5 following the last Example 10. A decrease in multiple genes encoding peroxidase expression suggests a decrease in the stress response that controls the cellular redox state. A decrease in GST expression is consistent with this. A decrease in expression of a WRKY transcription factor is similar to that seen with 1-MCP treatment. Additional analysis of the gene set reveals some patterns in gene expression, including a decrease in late embryogenesis abundant protein; β-glucosidase/copper ion binding/fucosidase/hydrolase, hydrolyzing O-glycosyl compounds; Vegetative Storage Protein; hydroxyproline-rich glycoprotein family protein; jacalin lectin family protein; Fatty Acid reductase. However, it is not known whether they are regulated by ethylene.

Table 4, following the last Example 10, lists informative Arabidopsis genes differentially expressed in response to COMPASS alone. The following Table 5 lists informative Arabidopsis genes common to either 1-MCP or COMPASS alone.

### EXAMPLE 10

### Gene Expression Induced by the Combination of 1-MCP and COMPASS Fungicide in Arabidopsis

Treatment of Arabidopsis with the combination of 1-methylcyclopropene and the COMPASS strobilurin fungicide resulted in the differential expression (≧5 fold change) of 93 genes on the microarray. From these, a subset of 44 genes unique to the combined treatment was further analyzed. For this analysis we focused on annotated genes and looked for changes/trends within related functional groups of genes. FIG. 2 shows the graphical results of the comparison of conditions.

The expression and putative function of these annotated genes is shown in Table 6. Although the gene set representing unique genes altered by the treatment with a combination of 1-MCP and strobulirin is small at 44 genes, some patterns in gene expression were observed including an increase in ethylene-responsive element-binding family protein (ERFs) and AP2 domain-containing transcription factors which belong to the ERF/AP2 superfamily of transcription factors. This data suggests that an increase in stress signaling may have occurred. The increase in expression of a disease resistance protein and C-Repeat-Binding Factor, which is a DNA binding transcription factor (note that CBF2 suppressed leaf senescence induced by the stress hormone ethylene) suggests alterations in stress signaling. The decrease in expression of another ethylene-responsive factor; a Pathogenesis-Related gene; AGD2-Like Defense Response Protein 1; and several heat shock protein suggests a possible decrease in stress responses. Thus, in Arabidopsis, the two compounds do not act antagonistically as in the corn results. This data provides evidence for a species and/or monocot/dicot specific response for the two compounds tested in these Examples. The following Table 6 lists informative Arabidopsis genes differentially expressed in response to 1-MCP and COMPASS fungicide in combination.

TABLE 1

| | Fold | Regu- | | |
|---|---|---|---|---|
| Probe Name | Change | lation | Hit_Description | Go_Consensus |
| ZmOL3043378 | 2.160074 | Up | Photosystem II subunit PsbS1 [Zea mays]. | Chloroplast photosystem ii 22 kda |
| ZmOL3020268 | 2.023517 | Up | 40S ribosomal protein S23 (RPS23B) [Arabidopsis | 30s ribosomal protein s12 |
| | | | thaliana)]. | |
| ZmOL3037849 | 2.121975 | Up | Os07g0616600 [Oryza sativa (japonica cultivar-group)]. | 30s ribosomal protein s10 |
| ZmOL3055816_2 | 2.289276 | Down | 60S ribosomal protein L17 [Picea mariana]. | 50s ribosomal protein l14 |
| ZmOL3050321_2 | 2.048178 | Up | RecName: Full = 30S ribosomal protein S1, chloroplastic; | 30s ribosomal protein s1 |
| | | | AltName: Full = CS1; Flags: Precursor. | |
| ZmOL3052137_2 | 2.120005 | Up | Os07g0616600 [Oryza sativa (japonica cultivar-group)]. | P40 (40s ribosomal protein sa) |
| ZmOL3014736 | 2.188196 | Up | Putative 60S ribosomal protein [Sorghum bicolor]. | |
| ZmOL3007616 | 2.929489 | Up | Os07g0660400 [Oryza sativa (japonica cultivar-group)]. | Ribosomal large subunit |
| ZmOL3043293 | 3.083715 | Up | Os06g0300600 [Oryza sativa (japonica cultivar-group)]. | Mitochondrial ribosomal protein |
| | | | | l46 |
| ZmOL3056321_2 | 2.94203 | Up | RL15_NEUCR 60S ribosomal protein L15 [Gibberella | |
| | | | zeae PH-1]. | |
| ZmOL3020067 | 2.022336 | Down | Gluthathione transferase24 [Zea mays]. | |
| ZmOL3032432 | 2. | Down | Thioredoxin/transketolase fusion protein [synthetic | Transketolase |
| | | | construct]. | |
| ZmOL3034759 | 2.007659 | Down | Thioredoxin h-like protein [Zea mays]. | Thioredoxi |
| ZmOL3026202 | 2.108909 | Down | Putative universal stress protein USP1 [Oryza sativa | Universal stress protein |
| | | | (indica cultivar-group)]. | |
| ZmOL3051436_2 | 2.25006 | Down | Drought inducible 22 kD protein [Saccharum | |
| | | | officinarum]. | |
| ZmOL3049418_2 | 2.005130 | Down | DNAJ heat shock N-termina43885760371218631 | Dnaj heat shock n-terminal |
| | | | domain-containing protein [Arabidopsis thaliana]. | domain-containing protein |
| ZmOL3007441 | 2.641916 | down | Os01g0733200 [Oryza sativa (japonica cultivar-group)]. | Heat shock transcription factor |
| ZmOL3000012 | 2.359727 | up | Beta-expansin 5 [Zea mays]. | Expansin |
| ZmOL3026229 | 2.015174 | up | Viviparous14 [Zea mays]. | 9-cis-epoxycarotenoid |
| | | | | dioxygenase |
| ZmOL3000174 | 2.05515 | up | Spermidine synthase [Zea mays]. | |
| ZmOL3027434 | 2.037929 | up | Os10g0155500 [Oryza sativa (japonica cultivar-group)]. | Aidose 1-epimerase |
| ZmOL3010863 | 2.384426 | up | Os04g0306400 [Oryza sativa (japonica cultivar-group)]. | Ribose-5-phosphate isomerase a |
| ZmOL3013329 | 2.331960 | up | NAD dependent epimerase/dehydratase family protein, | Short-chain dehydrogenase |
| | | | expressed [Oryza sativa (japonica cultivar-group)]. | reductase sdr |
| ZmOL3049240_2 | 2.021197 | up | Cinnamic acid 4-hydroxylase [Sorghum bicolor]. | |
| ZmOL3028816 | 2.539512 | up | ADP-glucose pyrophosphorylase small subunit [Zea | |
| | | | mays]. | |
| ZmOL3036555 | 2.000757 | up | Golgi associated protein se-wap41 [Zea mays]. | Reversibly glycosylated |
| | | | | polypeptide |
| ZmOL3006753 | 2.056715 | up | Putative blue copper protein precursor [Oryza sativa | Plastocyanin-like domain- |
| | | | Japonica Group]. | containing protein |
| ZmOL3057600_2 | 3.60271 | up | CBF3-like protein [Zea mays]. | Retrotransposonline subclass |
| ZmOL3042467 | 2.171900 | up | Ae1 protein [Zea mays]. | |
| ZmOL3045007_2 | 2.071408 | up | ClpP protease complex subunit ClpR3 [Arabidopsis | |
| | | | thaliana]. | |
| ZmOL3019515 | 2.154067 | up | 1-deoxy-D-xylulose 5-phosphate synthase 2 [Zea mays]. | 1-deoxy-d-xylulose-5-phosphate |
| ZmOL3055486_2 | 2.785253 | up | Lipase, putative, expressed [Oryza sativa (japonica | ---NA--- |
| | | | cultivar-group)]. | |
| ZmOL3027988 | 2.212391 | down | Putative UDP-glucose 4-epimerase [Oryza sativa | Udp-glucose 4-epimerase |
| | | | Japonica Group.] | |
| ZmOL3003141 | 2.39767 | down | Beta-amylase [Zea mays]. | Amyb_wheatame: full = beta- |
| | | | | amylaseame: full = alpha-d-glucan |
| ZmOL3034116 | 2.007233 | down | Carbonic anhydrase. | |
| mOL3034240 | 2.080644 | down | ATP synthase CFO A subunit [Triticum aestivum]. | |

TABLE 2

| | Fold | Regu- | | |
|---|---|---|---|---|
| Probe Name | Change | lation | Hit_Description | Go_Concensus |
| ZmOL3029450 | 2.1320302 | up | Putative 50S ribosomal protein L9, chloroplast [Oryza sativa | 50s ribosomal protein l9 |
| | | | Japonica Group]. | |
| ZmOL3035449 | 2.0095184 | up | OS03g0650700 [Oryza sativa (japonica cultivar-group)]. | Elongation factor 2 |
| ZmOL3054540_2 | 2.0018687 | up | Putative 60S ribosomal protein L39 [Oryza sativa Japonica | 50s ribosomal protein l39e |
| | | | Group]. | |
| ZmOL3042804 | 2.144698 | down | Putative plastid ribosomal protein L10 precursor [Oryza sativa]. | 50s ribosomal protein l19. |
| ZmOL3038502 | 2.8697567 | down | Ribosomal protein RL5 [Cicer arietinum]. | Pectinacetylesterase family |
| | | | | protein |
| ZmOL3050484_2 | 3.2523222 | down | Os10g0411800 [Oryza sativa (japonica cultivar-group)]. | 40s ribosomal protein s 17 |
| ZmOL3037047 | 2.3742876 | down | Putative eukaryotic translation initiation factor 4G [Oryza sativa | |
| | | | Japonica Group]. | |
| ZmOL3014561 | 2.4599373 | up | Glutaredoxin-like [Oryza sativa Japonica Group]. | Glutaredoxin family protein |
| ZmOL3031368 | 2.11545 | down | Thylakoid-bound ascorbate peroxidase [Triticum aestivum]. | |
| ZmOL3026676 | 8.213214 | down | Glutathione transferase5 [Zea mays]. | Glutathione s-transferase |
| ZmOL3049064_2 | 2.1016436 | down | Cytosolic glutathione reductase [Triticum monococcum]. | |
| ZmOL3054263_2 | 2.3786907 | down | Glutathione S-transferase2 [Zea Mays]. | |
| ZmOL3052873_2 | 2.101026 | up | Hsp70protein [Zea mays]. | Hsp 70 protein |
| ZmOL3044905_2 | 2.0124037 | up | Low temperature-induced protein 15 [Zea mays]. | |
| ZmOL3016160 | 2.1025667 | down | RecName: Full = Defensin-like protein 2; AltName: | |
| | | | Full = Gamma-zeathionin-2 | |
| ZmOL3010927 | 2.356285 | down | RecName: Full = Heat stress tramscription factor B-2b; AltName: | Heat shock transcription |
| | | | Full = Heat stress transcription factor 21: Short = OsHsf- | factor |
| | | | 21: AltName: Full = Heat stress transcription factor 2; | |
| | | | Short = rHsf2. | |
| ZmOL3037164 | 2.028073 | down | WRKY-like drought-induced protein [Retama raetam]. | Wrky dna-binding protein |
| ZmOL3043694 | 4.429324 | down | Temperature-induced lipocalin-1 [Zea mays]. | |
| ZmOL3003912 | 2.245548 | down | Phosphate starvation response regulator-like [Oryza sativa | Myb family transcription |
| | | | Japonica Group]. | factor |
| ZmOL3037909 | 2.2120402 | down | Retrotransposon protein, putative, unclassified [Oryza sativa | Disease resistance protein |
| | | | Japonica Group]. | |
| ZmOL3034101 | 2.3653462 | down | Barley mlo defense gene homolog4 [Zea mays]. | |
| ZmOL3019955 | 2.7665699 | down | Extension-like protein [Brassica napus]. | Protease inhibitor seed |
| | | | | storage lipid transfer protein |
| | | | | family protein |
| ZmOL3036348 | 2.742423 | up | Flavanone 3-hydroxylase 1 [Zea mays]/ | |
| ZmOL3015382 | 2.0833383 | up | Herbicide safener binding protein1 [Zea mays]. | o-methyltransferase |
| ZmOL3051635_2 | 2.203402 | up | Carbonic anhydrase [Zea mays]. | |
| ZmOL3056066_2 | 3.9435325 | down | Os07g0577600 [Oryza sativa (japonica cultivar-group)]. | Lhca44 protein of light- |
| | | | | harvesting complex of |
| | | | | photosystem i |
| ZmOL3018392 | 2.283792 | down | OSJNBa0041A02.19 [Oryza sativa (japonica cultivar-group)]. | Aidose 1-epimerase |
| ZmOL3028488 | 2.3240743 | down | Putative phosphofructokinase [Oryza sativa Japonica Group]. | |
| ZmOL3024430 | 2.3756304 | up | Histone deacetylase103 [Zea mays]. | |
| ZmOL3007191 | 2.0340362 | down | Histone deacetylase HDA101 [Zea mays]. | |
| ZmOL3011849 | 2.3495808 | down | OS03g0767000 [Oryza sativa (japonica cultivar-group)]. | Allene oxide synthase |
| ZmOL3003715 | 2.0176847 | up | OS01g0844500 [Oryza sativa (japonica cultivar-group)]. | Aspartyl protease family |
| | | | | protein |
| ZmOL3044123 | 2.218359 | down | Putative ominthine carbamoyltransferase [Oryza sativa Japonica | Ornithine |
| | | | group]. | carbamoyltransferase |

TABLE 3

| | Fold | Regu- | |
|---|---|---|---|
| ProbeName | change^{a} | lation^{a} | Description |
| A_84_P23570 | 5.575488 | up | Arabidopsis thaliana ACS5 (ACC SYNTHASE 5); 1-aminocyclopropane-1-carboxylate |
| | | | synthase (ACS5) mRNA, complete cds [NM_125977] - inhibited by wounding |
| A_84_P785139 | 9.926497 | up | Arabidopsis thaliana fatty acid desaturase family protein (AT1G06360) mRNA, complete |
| | | | cds [NM_100517] |
| A_84_P12289 | 5.803239 | up | Arabidopsis thaliana fatty acid desaturase family protein (AT1G06360) mRNA, complete |
| | | | cds [NM_100517] |
| A_84_P20410 | 5.772702 | up | Arabidopsis thaliana ADS1 (DELTA 9 DESATURASE 1); oxidoreductase (ADS1) mRNA, |
| | | | complete cds [NM_100489] |
| A_84_P196694 | 7.952991 | down | Arabidopsis thaliana PDF1.2c (plant defensin 1.2c) (PDF1.2c) mRNA, complete cds |
| | | | [NM_123810] |
| A_84_P239215 | 6.94666 | down | Arabidopsis thaliana PDF1.3 (plant defensin 1.3) (PDF1.3) mRNA, complete cds |
| | | | [NM_128160] |
| A_84_P137009 | 6.411213 | down | Arabidopsis thaliana PDF1.2 (PDF1.2) mRNA, complete cds [NM_123809] |
| A_84_P310613 | 7.147497 | down | Arabidopsis thaliana PDF1.2b (plant defensin 1.2b) (PDF1.2b) mRNA, complete cds |
| | | | [NM_128161] |
| A_84_P20061 | 9.013658 | down | Arabidopsis thaliana chitinase, putative (AT2G43580) mRNA, complete cds |
| | | | [NM_129920] |
| A_84_P15331 | 5.985378 | down | Arabidopsis thaliana chitinase, putative (AT2G43620) mRNA, complete cds |
| | | | [NM_129924] |
| A_84_P12287 | 6.4572062 | down | Arabidopsis thaliana ACS2; 1-aminocyclopropane-1-carboxylate synthase (ACS2) mRNA, |
| | | | complete cds [NM_100030] - induced by wounding |
| A_84_P558829 | 7.2555027 | down | Arabidopsis thaliana SAG13; alcohol dehydrogenase/oxidoreductase (SAG13) mRNA, |
| | | | complete cds [NM_201829] |
| A_84_P18335 | 8.455697 | down | Arabidopsis thaliana ABA-responsive protein-related (AT3G02480) mRNA, complete cds |
| | | | [NM_111115] |
| A_84_P588071 | 6.021794 | down | Arabidopsis thaliana early-responsive to dehydration protein-related/ERD protein- |
| | | | related (AT1G62320) mRNA, complete cds [NM_104912] |
| A_84_P10637 | 18.525372 | down | Arabidopsis thaliana GEA6 (LATE EMBRYOGENESIS ABUNDANT 6) (GEA6) mRNA, |
| | | | complete cds [NM_129575] |
| A_84_P19215 | 9.743442 | down | Arabidopsis thaliana DNAJ heat shock N-terminal domain-containing protein (AT2G21510) |
| | | | mRNA, complete cds [NM_127723] |
| A_84_P15440 | 6.1465635 | down | Arabidopsis thaliana 17.8 kDa class I heat shock protein (HSP17.8-CI) (AT1G07400) |
| | | | mRNA, complete cds [NM_100614] |
| A_84_P582388 | 6.3814926 | down | Arabidopsis thaliana universal stress protein (USP) family protein (AT5G17390) mRNA, |
| | | | complete cds [NM_121745] |
| A_84_P15731 | 10.176264 | down | Arabidopsis thaliana ATHSP23.6-MITO (MITOCHONDRION-LOCALIZED SMALL HEAT |
| | | | SHOCK PROTEIN 23.6) (ATHSP23.6-MITO) mRNA, complete cds [NM_118652] |
| A_84_P11264 | 13.637636 | down | Arabidopsis thaliana ATGSTU9 (ARABIDOPSIS THALIANA GLUTATHIONE |
| | | | S-TRANSFERASE TAU 9); glutathione transferase (ATGSTU9) mRNA, complete cds |
| | | | [NM_125642] |
| A_84_P18100 | 5.4993687 | down | Arabidopsis thaliana ATGSTU11 (GLUTATHIONE S-TRANSFERASE TAU 11); |
| | | | glutathione transferase (ATGSTU11) mRNA, complete cds [NM_105661] |
| A_84_P23724 | 10.569552 | down | Arabidopsis thaliana ATGSTU25 (GLUTATHIONE S-TRANSFERASE TAU 25); |
| | | | glutathione transferase (ATGSTU25) mRNA, complete cds [NM_101579] |
| A_84_P14441 | 6.336314 | down | Arabidopsis thaliana ATGSTU4 (ARABIDOPSIS THALIANA GLUTATHIONE |
| | | | S-TRANSFERASE TAU 4); glutathione transferase (ATGSTU4) mRNA, complete cds |
| | | | [NM_128500] |
| A_84_P12432 | 5.087074 | down | Arabidopsis thaliana GSTU10 (GLUTATHIONE S-TRANSFERASE TAU 10); glutathione |
| | | | transferase (GSTU10) mRNA, complete cds [NM_106118] |
| A_84_P290004 | 5.900666 | down | Arabidopsis thaliana methionine sulfoxide reductase domain-containing protein/SeIR |
| | | | domain-containing protein (AT4G21850) mRNA, complete cds [NM_179087] |
| A_84_P812152 | 5.9906445 | down | Arabidopsis thaliana methionine sulfoxide reductase domain-containing protein/SeIR |
| | | | domain-containing protein (AT4G21850) mRNA, complete cds [NM_118305] |
| A_84_P125591 | 11.801208 | down | Arabidopsis thaliana methionine sulfoxide reductase domain-containing protein/SeIR |
| | | | domain-containing protein (AT4G21830) mRNA, complete cds [NM_118303] |
| A_84_P106016 | 10.988058 | down | Arabidopsis thaliana S-adenosylmethionine-dependent methyltransferase/methyltransferase |
| | | | (AT1G15125) mRNA, complete cds [NM_148466] |
| A_84_P836406 | 5.2474995 | down | Arabidopsis thaliana S-adenosylmethionine-dependent methyltransferase/methyltransferase |
| | | | (AT1G15125) mRNA, complete cds [NM_148466] |
| A_84_P829373 | 14.167934 | down | Arabidopsis thaliana MAPKKK19; ATP binding/kinase/protein kinase/protein |
| | | | serine/threonine kinase (MAPKKK19) mRNA, complete cds [NM_126108] |
| A_84_P13172 | 12.065134 | down | Arabidopsis thaliana MAPKKK19; ATP binding/kinase/protein kinase/protein |
| | | | serine/threonine kinase (MAPKKK19) mRNA, complete cds [NM_126108] |
| A_84_P762988 | 5.2091174 | down | Arabidopsis thaliana MAPKKK21; ATP binding/protein kinase/protein serine/threonine |
| | | | kinase (MAPKKK21) mRNA, complete cds [NM_001085035] |
| A_84_P11148 | 7.6739216 | down | Arabidopsis thaliana protein kinase family protein (AT5G24080) mRNA, complete cds |
| | | | [NM_122313] |
| A_84_P17614 | 5.0515103 | down | Arabidopsis thaliana protein kinase family protein (CRK13) mRNA, complete cds |
| | | | [NM_001084966] |
| A_84_P566529 | 7.117308 | down | Arabidopsis thaliana protein kinase family protein (AT5G41730) mRNA, complete cds |
| | | | [NM_123538] |
| A_84_P286390 | 5.872223 | down | Arabidopsis thaliana AOC3 (ALLENE OXIDE CYCLASE 3); allene-oxide cyclase (AOC3) |
| | | | mRNA, complete cds [NM_113477] - catalyzes the first step in the biosynthesis of jasmonic |
| | | | acid - Ethylene induces the expression of AOS |
| A_84_P825539 | 13.780353 | down | Arabidopsis thaliana AOX1D (alternative oxidase 1D); alternative oxidase (AOX1D) |
| | | | mRNA, complete cds [NM_102968] |
| A_84_P21945 | 7.2751045 | down | Arabidopsis thaliana AOX1D (alternative oxidase 1D); alternative oxidase (AOX1D) |
| | | | mRNA, complete cds [NM_102968] |
| A_84_P10183 | 5.7478347 | down | Arabidopsis thaliana peroxidase, putative (AT5G14130) mRNA, complete cds |
| | | | [NM_121417] |
| A_84_P16626 | 8.399267 | down | Arabidopsis thaliana peroxidase, putative (AT4G08770) mRNA, complete cds |
| | | | [NM_116947] |
| A_84_P19375 | 15.481089 | down | Arabidopsis thaliana PRXCA (PEROXIDASE CA); peroxidase (PRXCA) mRNA, complete |
| | | | cds [NM_114770] |
| A_84_P18528 | 5.2329216 | down | Arabidopsis thaliana peroxidase, putative (AT4G11290) mRNA, complete cds |
| | | | [NM_117200] |
| A_84_P22012 | 6.336872 | down | Arabidopsis thaliana MYB14 (MYB DOMAIN PROTEIN 14); DNA binding/transcription |
| | | | factor (MYB14) mRNA, complete cds [NM_128674] |
| A_84_P22140 | 9.846942 | down | Arabidopsis thaliana MYB15 (MYB DOMAIN PROTEIN 15); DNA binding/transcription |
| | | | factor (MYB15) mRNA, complete cds [NM_001035670] |
| A_84_P870349 | 9.9232 | down | Arabidopsis thaliana MYB15 (MYB DOMAIN PROTEIN 15); DNA binding/transcription |
| | | | factor (MYB15) mRNA, complete cds [NM_001035670] |
| A_84_P22549 | 6.7267246 | down | Arabidopsis thaliana WRKY8; transcription factor (WRKY8) mRNA, complete cds |
| | | | [NM_124005] some known to be induced, others repressed by ethylene |
| A_84_P786837 | 9.454134 | down | Arabidopsis thaliana WRKY75; transcription factor (WRKY75) mRNA, complete cds |
| | | | [NM_121311] |
| A_84_P786760 | 5.530835 | down | Arabidopsis thaliana WRKY40; transcription factor (WRKY40) mRNA, complete cds |
| | | | [NM_106732] |
| A_84_P13271 | 5.5672174 | down | Arabidopsis thaliana WRKY40; transcription factor (WRKY40) mRNA, complete cds |
| | | | [NM_106732] |
| A_84_P21703 | 8.746374 | down | Arabidopsis thaliana WRKY75; transcription factor (WRKY75) mRNA, complete cds |
| | | | [NM_121311] |
| A_84_P75404 | 7.271174 | down | Arabidopsis thaliana F-box family protein (AT5G55150) mRNA, complete cds |
| | | | [NM_124897] some F-box proteins down regulated by ethylene such as those regulating EIN3 |
| A_84_P533104 | 6.482149 | down | Arabidopsis thaliana F-box family protein (AT2G14290) mRNA, complete cds |
| | | | [NM_126996] |

TABLE 4

| | Fold | Regu- | |
|---|---|---|---|
| ProbeName | change^{a} | lation^{a} | Description |
| A_84_P806966 | 5.0941978 | up | Arabidopsis thaliana ATEXPA8 (ARABIDOPSIS THALIANA EXPANSIN A8) (ATEXPA8) |
| | | | mRNA, complete cds [NM_129623] |
| A_84_P13426 | 5.13506 | up | Arabidopsis thaliana CYP96A15 (CYTOCHROME P450 96 A1); midchain alkane |
| | | | hydroxylase/oxygen binding (CYP96A15) mRNA, complete cds [NM_104570] |
| A_84_P20466 | 12.762498 | up | Arabidopsis thaliana MIOX4; inositol oxygenase (MIOX4) mRNA, complete cds |
| | | | [NM_118759] |
| A_84_P811407 | 5.918804 | up | Arabidopsis thaliana DIN10 (DARK INDUCIBLE 10); hydrolase, hydrolyzing O-glycosyl |
| | | | compounds (DIN10) mRNA, complete cds [NM_122032] |
| A_84_P853466 | 9.361319 | up | Arabidopsis thaliana DIN10 (DARK INDUCIBLE 10); hydrolase, hydrolyzing O-glycosyl |
| | | | compounds (DIN10) mRNA, complete cds [NM_122032] |
| A_84_P805621 | 10.093518 | down | Arabidopsis thaliana VSP1 (VEGETATIVE STORAGE PROTEIN 1); acid phosphatase/ |
| | | | transcription factor binding (VSP1) mRNA, complete cds [NM_001125801] |
| A_84_P141439 | 13.1223 | down | Arabidopsis thaliana VSP2 (VEGETATIVE STORAGE PROTEIN 2); acid phosphatase |
| | | | (VSP2) mRNA, complete cds [NM_122386] |
| A_84_P808818 | 11.950743 | down | Arabidopsis thaliana VSP2 (VEGETATIVE STORAGE PROTEIN 2); acid phosphatase |
| | | | (VSP2) mRNA, complete cds [NM_001036860] |
| A_84_P524680 | 6.3855186 | down | Arabidopsis thaliana JAZ10 (JASMONATE-ZIM-DOMAIN PROTEIN 10) (JAZ10) mRNA, |
| | | | complete cds [NM_203046] |
| A_84_P823221 | 5.5852847 | down | Arabidopsis thaliana JAZ10 (JASMONATE-ZIM-DOMAIN PROTEIN 10) (JAZ10) mRNA, |
| | | | complete cds [NM_121325] |
| A_84_P105636 | 5.3258333 | down | Arabidopsis thaliana NSP1 (NITRILE SPECIFIER PROTEIN 1) (NSP1) mRNA, complete cds |
| | | | [NM_112511] Nitrile-specifier proteins involved in glucosinolate hydrolysis involved in |
| | | | defense |
| A_84_P15355 | 7.182606 | down | Arabidopsis thaliana NSP2 (NITRILE SPECIFIER PROTEIN 2) (NSP2) mRNA, complete cds |
| | | | [NM_128867] |
| A_84_P812141 | 5.3820753 | down | Arabidopsis thaliana NSP1 (NITRILE SPECIFIER PROTEIN 1) (NSP1) mRNA, complete cds |
| | | | [NM_112511] |
| A_84_P812128 | 5.764313 | down | Arabidopsis thaliana NSP1 (NITRILE SPECIFIER PROTEIN 1) (NSP1) mRNA, complete cds |
| | | | [NM_112511] |
| A_84_P21473 | 6.406524 | down | Arabidopsis thaliana BAM5 (BETA-AMYLASE 5); beta-amylase (BAM5) mRNA, complete |
| | | | cds [NM_117609] |
| A_84_P567982 | 5.096871 | down | Arabidopsis thaliana BAM5 (BETA-AMYLASE 5); beta-amylase (BAM5) mRNA, complete |
| | | | cds [NM_117609] |
| A_84_P22843 | 15.196865 | down | Arabidopsis thaliana O-methyltransferase family 2 protein (AT1G76790) mRNA, complete cds |
| | | | [NM_106329] |
| A_84_P858400 | 16.490932 | down | Arabidopsis thaliana O-methyltransferase family 2 protein (AT1G76790) mRNA, complete cds |
| | | | [NM_106329] |
| A_84_P20921 | 5.6124716 | down | Arabidopsis thaliana O-methyltransferase family 2 protein (AT1G77520) mRNA, complete cds |
| | | | [NM_106401] |
| A_84_P502791 | 7.470893 | down | Arabidopsis thaliana transferase family protein (AT1G32910) mRNA, complete cds |
| | | | [NM_103024] |
| A_84_P22519 | 5.1034837 | down | Arabidopsis thaliana transferase family protein (AT5G38130) mRNA, complete cds |
| | | | [NM_123173] |
| A_84_P22640 | 9.273502 | down | Arabidopsis thaliana transferase family protein (AT5G17540) mRNA, complete cds |
| | | | [NM_121760] |
| A_84_P853518 | 8.5159 | down | Arabidopsis thaliana JR1 (JR1) mRNA, complete cds [NM_112518] - wound-responsive |
| | | | genes induced by JA |
| A_84_P15536 | 8.934541 | down | Arabidopsis thaliana JR1 (JR1) mRNA, complete cds [NM_112518] |
| A_84_P564785 | 5.567845 | down | Arabidopsis thaliana nucleoside phosphatase family protein/GDA1/CD39 family protein |
| | | | (AT1G14250) mRNA, complete cds [NM_101291] |
| A_84_P12373 | 5.2832813 | down | Arabidopsis thaliana nucleoside phosphatase family protein/GDA1/CD39 family protein |
| | | | (AT1G14240) mRNA, complete cds [NM_001123812] |
| A_84_P831354 | 9.226337 | down | Arabidopsis thaliana palmitoyl protein thioesterase family protein (AT4G17470) mRNA, |
| | | | complete cds [NM_117850] |
| A_84_P542288 | 9.371448 | down | Arabidopsis thaliana palmitoyl protein thioesterase family protein (AT4G17470) mRNA, |
| | | | complete cds [NM_117850] |
| A_84_P19474 | 5.534677 | down | Arabidopsis thaliana cysteine proteinase, putative (AT4G11310) mRNA, complete cds |
| | | | [NM_117202] |
| A_84_P851624 | 15.136853 | down | Arabidopsis thaliana cysteine proteinase, putative (AT4G11320) mRNA, complete cds |
| | | | [NM_117203] |
| A_84_P112392 | 5.5426965 | down | Arabidopsis thaliana NAI2 (NAI2) mRNA, complete cds [NM_112465] |
| A_84_P861607 | 5.649866 | down | Arabidopsis thaliana NAI2 (NAI2) mRNA, complete cds [NM_112465] |

TABLE 5

| | Fold | Regu- | Fold | Regu- | |
|---|---|---|---|---|---|
| ProbeName | change^{a} | lation^{a} | change^{b} | lation^{b} | Description |
| A_84_P16177 | 8.121181 | up | 5.7452345 | Up | Arabidopsis thaliana ATGA2OX1 (gibberellin 2-oxidase 1); |
| | | | | | gibberellin 2-beta-dioxygenase (ATGA2OX1) mRNA, complete cds |
| | | | | | [NM_106491] |
| A_84_P22610 | 6.873894 | up | 5.458554 | Up | Arabidopsis thaliana embryo-specific protein-related (AT5G62210) |
| | | | | | mRNA, complete cds [NM_125615] |
| A_84_P507728 | 5.6376114 | up | 5.314121 | Up | Arabidopsis thaliana SP1L5 (SPIRAL1-LIKE5) (SP1L5) mRNA, |
| | | | | | complete cds [NM_118480] |
| A_84_P19553 | 5.504292 | up | 5.559119 | Up | Arabidopsis thaliana CER4 (ECERIFERUM 4); fatty acyl-CoA |
| | | | | | reductase (alcohol-forming)/oxidoreductase, acting on the CH-CH |
| | | | | | group of donors (CER4) mRNA, complete cds [NM_119537] |
| A_84_P64534 | 8.048617 | up | 5.9451394 | Up | Arabidopsis thaliana glucose-methanol-choline (GMC) |
| | | | | | oxidoreductase family protein (AT1G12570) mRNA, complete cds |
| | | | | | [NM_101128] |
| A_84_P13916 | 5.4405384 | down | 6.528005 | down | Arabidopsis thaliana peroxidase, putative (AT4G36430) mRNA, |
| | | | | | complete cds [NM_119806] |
| A_84_P11481 | 22.2079 | down | 17.507862 | down | Arabidopsis thaliana peroxidase, putative (AT1G68850) mRNA, |
| | | | | | complete cds [NM_105559] |
| A_84_P18663 | 20.755585 | down | 22.14512 | down | Arabidopsis thaliana peroxidase, putative (AT5G05340) mRNA, |
| | | | | | complete cds [NM_120616] |
| A_84_P22055 | 11.450264 | down | 15.756354 | down | Arabidopsis thaliana peroxidase 22 (PER22) (P22) (PRXEA)/ |
| | | | | | basic peroxidase E (AT2G38380) mRNA, complete cds |
| | | | | | [NM_129394] |
| A_84_P14274 | 11.51948 | down | 5.444658 | down | Arabidopsis thaliana late embryogenesis abundant protein, |
| | | | | | putative/LEA protein, putative (AT1G52690) mRNA, complete cds |
| | | | | | [NM_202280] |
| A_84_P22906 | 14.128903 | down | 5.529438 | down | Arabidopsis thaliana late embryogenesis abundant domain- |
| | | | | | containing protein/LEA domain-containing protein (AT2G42560) |
| | | | | | mRNA, complete cds [NM_129817] |
| A_84_P810225 | 10.831495 | down | 25.469591 | down | Arabidopsis thaliana PYK10; beta-glucosidase/copper ion binding/ |
| | | | | | fucosidase/hydrolase, hydrolyzing O-glycosyl compounds |
| | | | | | (PYK10) mRNA, complete cds [NM_111760] |
| A_84_P861135 | 6.473847 | down | 8.508572 | down | Arabidopsis thaliana PYK10; beta-glucosidase/copper ion binding/ |
| | | | | | fucosidase/hydrolase, hydrolyzing O-glycosyl compounds |
| | | | | | (PYK10) mRNA, complete cds [NM_111760] |
| A_84_P22436 | 9.84361 | down | 5.947356 | down | Arabidopsis thaliana WRKY62; transcription factor (WRKY62) |
| | | | | | mRNA, complete cds [NM_120268] |
| A_84_P784216 | 7.327667 | down | 18.497337 | down | Arabidopsis thaliana VSP1 (VEGETATIVE STORAGE PROTEIN |
| | | | | | 1); acid phosphatase/transcription factor binding (VSP1) mRNA, |
| | | | | | complete cds [NM_001125801] |
| A_84_P97476 | 7.04099 | down | 17.203436 | down | Arabidopsis thaliana VSP1 (VEGETATIVE STORAGE PROTEIN |
| | | | | | 1); acid phosphatase/transcription factor binding (VSP1) mRNA, |
| | | | | | complete cds [NM_122387] |
| A_84_P805566 | 7.2282424 | down | 18.194885 | down | Arabidopsis thaliana VSP1 (VEGETATIVE STORAGE PROTEIN |
| | | | | | 1); acid phosphatase/transcription factor binding (VSP1) mRNA, |
| | | | | | complete cds [NM_122387] |
| A_84_P863273 | 44.06568 | down | 66.262024 | down | Arabidopsis thaliana hydroxyproline-rich glycoprotein family protein |
| | | | | | (AT5G09530) mRNA, complete cds [NM_120990] - in some |
| | | | | | species, induced by wounding |
| A_84_P861864 | 10.505951 | down | 8.861952 | down | Arabidopsis thaliana hydroxyproline-rich glycoprotein family protein |
| | | | | | (AT5G09530) mRNA, complete cds [NM_120990] |
| A_84_P13969 | 7.877567 | down | 5.7286553 | down | Arabidopsis thaliana ATGSTF12 (ARABIDOPSIS THALIANA |
| | | | | | GLUTATHIONE S-TRANSFERASE PHI 12); glutathione |
| | | | | | transferase (ATGSTF12) mRNA, complete cds [NM_121728] |
| A_84_P817628 | 9.525247 | down | 9.984217 | down | Arabidopsis thaliana JAL23 (JACALIN-RELATED LECTIN 23) |
| | | | | | (JAL23) mRNA, complete cds [NM_129490] |
| A_84_P10711 | 15.099809 | down | 17.30689 | down | Arabidopsis thaliana JAL23 (JACALIN-RELATED LECTIN 23) |
| | | | | | (JAL23) mRNA, complete cds [NM_129490] |
| A_84_P12602 | 6.7814097 | down | 14.20866 | down | Arabidopsis thaliana JAL22 (JACALIN-RELATED LECTIN 22) |
| | | | | | (JAL22) mRNA, complete cds [NM_129488] |
| A_84_P792597 | 5.6843925 | down | 8.564349 | down | Arabidopsis thaliana jacalin lectin family protein (AT3G16450) |
| | | | | | mRNA, complete cds [NM_180265] |
| A_84_P219878 | 5.630177 | down | 7.3030233 | down | Arabidopsis thaliana jacalin lectin family protein (AT3G16450) |
| | | | | | mRNA, complete cds [NM_180265] |
| A_84_P810350 | 7.464595 | down | 5.652608 | down | Arabidopsis thaliana jacalin lectin family protein (AT3G16450) |
| | | | | | mRNA, complete cds [NM_180265] |
| A_84_P740321 | 13.871204 | down | 8.559692 | down | Arabidopsis thaliana FAR5 (FATTY ACID REDUCTASE 5); binding/ |
| | | | | | catalytic/oxidoreductase, acting on the CH-CH group of donors |
| | | | | | (FAR5) mRNA, complete cds [NM_114323] |
| A_84_P787920 | 8.893051 | down | 5.8567753 | down | Arabidopsis thaliana FAR5 (FATTY ACID REDUCTASE 5); binding/ |
| | | | | | catalytic/oxidoreductase, acting on the CH-CH group of donors |
| | | | | | (FAR5) mRNA, complete cds [NM_114323] |
| A_84_P19356 | 16.227991 | down | 21.525135 | down | Arabidopsis thaliana FAR4 (FATTY ACID REDUCTASE 4); binding/ |
| | | | | | catalytic/oxidoreductase, acting on the CH-CH group of donors |
| | | | | | (FAR4) mRNA, complete cds [NM_001035732] |

TABLE 6

| | Fold | Regu- | |
|---|---|---|---|
| ProbeName | change^{a} | lation^{a} | Description |
| A_84_P22607 | 5.7642283 | up | Arabidopsis thaliana ethylene-responsive element-binding family protein (AT5G61600) mRNA, |
| | | | complete cds [NM_125553] |
| A_84_P820417 | 7.1169624 | up | Arabidopsis thaliana ATERF6 (ETHYLENE RESPONSIVE ELEMENT BINDING FACTOR 6); |
| | | | DNA binding/transcription factor (ATERF6) mRNA, complete cds [NM_117854] |
| A_84_P11223 | 7.8111267 | up | Arabidopsis thaliana AP2 domain-containing protein (AT5G52020) mRNA, complete cds |
| | | | [NM_124581] - ethylene response factor/Apetala2 family |
| A_84_P296724 | 10.317429 | up | Arabidopsis thaliana AP2 domain-containing transcription factor, putative (AT5G51190) |
| | | | mRNA, complete cds [NM_124498] |
| A_84_P18908 | 28.56238 | up | Arabidopsis thaliana AP2 domain-containing transcription factor, putative (AT1G33760) |
| | | | mRNA, complete cds [NM_103095] |
| A_84_P512655 | 8.181378 | up | Arabidopsis thaliana AP2 domain-containing transcnption factor, putative (AT1G77640) |
| | | | mRNA, complete cds [NM_106412] |
| A_84_P20976 | 7.197986 | up | Arabidopsis thaliana AP2 domain-containing transcription factor, putative (AT1G19210) |
| | | | mRNA, complete cds [NM_101779] |
| A_84_P15185 | 5.6828427 | up | Arabidopsis thaliana AP2 domain-containing transcription factor, putative (AT1G22810) |
| | | | mRNA, complete cds [NM_102128] |
| A_84_P785348 | 10.051393 | up | Arabidopsis thaliana AP2 domain-containing transcription factor, putative (AT5G51190) |
| | | | mRNA, complete cds [NM_124498] |
| A_84_P20462 | 6.8735604 | up | Arabidopsis thaliana CBF1 (C-REPEAT/DRE BINDING FACTOR 1); DNA binding/ |
| | | | transcription activator/transcription factor (CBF1) mRNA, complete cds [NM_118681] - |
| A_84_P23516 | 7.565255 | up | Arabidopsis thaliana CBF4 (C- REPEAT-BINDING FACTOR 4); DNA binding/transcription |
| | | | activator/transcription factor (CBF4) mRNA, complete cds [NM_124578] - CBF2 suppressed |
| | | | leaf senescence induced by the stress hormone ethylene |
| A_84_P819549 | 5.061593 | up | Arabidopsis thaliana disease resistance protein (TIR-NBS-LRR class), putative (AT5G41740) |
| | | | mRNA, complete cds [NM_123539] |
| A_84_P16247 | 6.2125435 | up | Arabidopsis thaliana DDF1 (DWARF AND DELAYED FLOWERING 1); DNA binding/ |
| | | | sequence-specific DNA binding/transcription factor (DDF1) mRNA, complete cds |
| | | | [NM_101131] |
| A_84_P784166 | 7.378656 | up | Arabidopsis thaliana CYP707A3; (+)-abscisic acid 8′-hydroxylase/oxygen binding (CYP707A3) |
| | | | mRNA, complete cds [NM_123902] |
| A_84_P594936 | 6.453371 | up | Arabidopsis thaliana CYP707A3; (+)-abscisic acid 8′-hydroxylase/oxygen binding (CYP707A3) |
| | | | mRNA, complete cds [NM_123902] |
| A_84_P12143 | 7.712123 | up | Arabidopsis thaliana CYP707A3; (+)-abscisic acid 8′-hydroxylase/oxygen binding (CYP707A3) |
| | | | mRNA, complete cds [NM_180805] |
| A_84_P18566 | 7.023545 | up | Arabidopsis thaliana mitochondrial substrate carrier family protein (AT4G24570) mRNA, |
| | | | complete cds [NM_118590] |
| A_84_P15741 | 5.1320925 | up | Arabidopsis thaliana calcium-binding EF hand family protein (AT4G27280) mRNA, complete |
| | | | cds [NM_118862] |
| A_84_P17859 | 5.385682 | up | Arabidopsis thaliana TCH4 (Touch 4); hydrolase, acting on glycosyl bonds/ |
| | | | xyloglucan:xyloglucosyl transferase (TCH4) mRNA, complete cds (NM_125137] |
| A_84_P17191 | 7.802257 | up | Arabidopsis thaliana protein phosphatase 2C, putative/PP2C, putative (AT1G07160) mRNA, |
| | | | complete cds [NM_100590] |
| A_84_P16799 | 6.6954465 | up | Arabidopsis thaliana atnudt4 (Arabidopsis thaliana Nudix hydrolase homolog 4); hydrolase |
| | | | (atnudt4) mRNA, complete cds [NM_101688] |
| A_84_P21946 | 5.938087 | up | Arabidopsis thaliana atnudt21 (Arabidopsis thaliana Nudix hydrolase homolog 21); hydrolase |
| | | | (atnudt21) mRNA, complete cds [NM_106012] |
| A_84_P14970 | 5.224872 | down | Arabidopsis thaliana ethylene-responsive factor, putative (AT5G43410) mRNA, complete cds |
| | | | [NM_123707] |
| A_84_P17268 | 6.8723383 | down | Arabidopsis thaliana PR1 (PATHOGENESIS-RELATED GENE 1) (PR1) mRNA, complete cds |
| | | | [NM_127025] |
| A_84_P23968 | 5.8652425 | down | Arabidopsis thaliana ALD1 (AGD2-LIKE DEFENSE RESPONSE PROTEIN1); catalytic/ |
| | | | pyridoxal phosphate binding/transaminase/transferase, transferring n_ (ALD1) mRNA, |
| | | | complete cds [NM_126957] |
| A_84_P21525 | 6.2565274 | down | Arabidopsis thaliana AT-HSP17.6A (ARABIDOPSIS THALIANA HEAT SHOCK PROTEIN |
| | | | 17.6A); unfolded protein binding (AT-HSP17.6A) mRNA, complete cds [NM_121241] |
| A_84_P20579 | 5.2758956 | down | Arabidopsis thaliana HSP17.6II (17.6 KDA CLASS II HEAT SHOCK PROTEIN) (HSP17.6II) |
| | | | mRNA, complete cds [NM_121240] |
| A_84_P19363 | 9.78248 | down | Arabidopsis thaliana ATHSP17.4 (ATHSP17.4) mRNA, complete cds [NM_114492] |
| A_84_P23899 | 5.1303573 | down | Arabidopsis thaliana 17.6 kDa class I small heat shock protein (HSP17.6B-CI) (AT2G29500) |
| | | | mRNA, complete cds [NM_128504] |
| A_84_P22839 | 5.697062 | down | Arabidopsis thaliana trypsin and protease inhibitor family protein/Kunitz family protein |
| | | | (AT1G73260) mRNA, complete cds [NM_105985] |
| A_84_P832617 | 5.6128454 | down | Arabidopsis thaliana CYP79A3P; electron carrier/heme binding/iron ion binding/ |
| | | | monooxygenase (CYP79A3P) mRNA, complete cds [NM_148046] |
| A_84_P22718 | 6.270766 | down | Arabidopsis thaliana SPP1 (SUCROSE-PHOSPHATASE 1); catalytic/magnesium ion binding/ |
| | | | phosphatase/sucrose-phosphatase (SPP1) mRNA, complete cds [NM_104020] |
| A_84_P11129 | 5.557714 | down | Arabidopsis thaliana MPL1 (MYZUS PERSICAE-INDUCED LIPASE 1); catalytic (MPL1) |
| | | | mRNA, complete cds [NM_121422] |
| A_84_P19342 | 5.439648 | down | Arabidopsis thaliana oxidoreductase, 2OG-Fe(II) oxygenase family protein (AT3G13610) |
| | | | mRNA, complete cds [NM_112207] |
| A_84_P859968 | 5.072212 | down | Arabidopsis thaliana MPL1 (MYZUS PERSICAE-INDUCED LIPASE 1); catalytic (MPL1) |
| | | | mRNA, complete cds [NM_121422] |
| A_84_P833880 | 6.7572927 | down | Arabidopsis thaliana late embryogenesis abundant protein-related/LEA protein-related |
| | | | (AT1G54890) mRNA, complete cds [NM_104362] |
| A_84_P586394 | 8.143425 | down | Arabidopsis thaliana protein binding/structural molecule (AT2G16200) mRNA, complete cds |
| | | | [NM_127177] |
| A_84_P21916 | 6.115354 | down | Arabidopsis thaliana FMO1 (FLAVIN-DEPENDENT MONOOXYGENASE 1); FAD binding/ |
| | | | NADP or NADPH binding/electron carrier/flavin-containing monooxygena_ (FMO1) mRNA, |
| | | | complete cds [NM_101783] |
| A_84_P12640 | 7.9059653 | down | Arabidopsis thaliana DOX1; lipoxygenase (DOX1) mRNA, complete cds [NM_111008] |

All documents, including patents, patent applications and publications, and non-patent publications listed or referred to above, as well as the attached figures are incorporated herein by reference in their entireties to the extent they are not inconsistent with the explicit teachings of this specification. However, the citation of any reference herein should not be construed as an admission that such reference is available as “Prior Art” to the instant application.

While various embodiments in the specification or claims are presented using “comprising” language, under various circumstances, a related embodiment may also be described using “consisting of” or “consisting essentially of” language. It is to be noted that the term “a” or “an”, refers to one or more, for example, “a compound,” is understood to represent one or more compounds. As such, the terms “a” (or “an”), “one or more,” and “at least one” are used interchangeably herein. The invention has been described with reference to specific embodiments and examples. However, it is appreciated that modifications can be made without departing from the spirit of the invention. Such modifications are intended to fall within the scope of the appended claims.

What is claimed is:

1. A method for identifying compounds that selectively alter wildtype gene expression of a plant cell, the method comprising:
(a) comparing
(i) a first expression profile from a plant cell or tissue contacted with a first test compound,
(ii) an additional expression profile from the same plant cell or tissue contacted with an additional test compound, and
(iii) a combined compound expression profile from the same plant cell or tissue contacted with the first test compound and the additional test compound,
wherein each plant cell or tissue was contacted for a time sufficient to produce an alteration in the expression of at least one gene or gene product in the plant's cells or tissues; and wherein each expression profile comprises the expression level or pattern of at least one gene or gene product of the plant cell or tissue; and
(b) identifying a significant, unexpected alteration in the level or pattern of expression between the genes or gene products of the combined compound profile and those of the additive alterations of the first profile and the additional profile.

2. The method according to claim 1, further comprising the steps of:
(c) contacting a plant cell or tissue from the same plant with one of:
(i) a first test compound;
(ii) an additional test compound; or
(iii) the combination of the first test compound and the additional test compound; and
(d) generating
(i) a first expression profile from the plant cell or tissue contacted with (c)(i);
(ii) an additional expression profile from the plant cell or tissue contacted with (c)(ii); and
(iii) a combined compound expression profile from the plant cell or tissue of (c)(iii).

3. The method according to claim 1, wherein wherein the alteration in expression of the contacted plant cell's genes or gene products in the combined compound profile from those of the additive alterations of the first profile and the additional profile correlates with a selected effect on the plant cell caused by the compounds in combination.

4. The method according to claim 1, wherein a wildtype gene or gene product expression profile from the cells or tissues of the same plant, which is not contacted with either the first or an additional test compound, is subtracted from each profile prior to the comparing step.

5. The method according to claim 1, wherein the alteration in the expression profile is expressed as a mean or average, a numerical mean or range of numerical means, a numerical pattern, a graphical pattern, or an expression profile.

6. The method according to claim 1, wherein said alteration is selected from the group consisting of:
(i) an upregulation of the same selected gene or gene product in the combined compound profile compared with the additive expression of the same gene or gene product in the first profile and the additional profile; and
(ii) a downregulation of the same selected gene or gene product in the combined compound profile compared with the additive expression of the same gene or gene product in the first profile and the additional profile; and
(iii) a change in identity or expression of one or more selected gene or gene product in the combined compound profile which selected gene or gene product was unaltered in a profile generated from the additive alterations of the first profile and the additional profile.

7. The method according to claim 1, wherein the first compound has a known effect on the plant and wherein the additional compound has an unknown effect on the plant.

8. The method according to claim 1, wherein each expression profile comprises a subset of genes or gene products encoded by the complete genome of the plant.

9. The method according to claim 1, wherein the gene product is a plant protein.

10. The method according to claim 1, further comprising identifying a corresponding change in a plant metabolite as a result of the alteration of a gene product.

11. The method according to claim 1, wherein the combination of compounds has a synergistic effect that is a greater alteration in expression patterns or levels of a gene or gene product of the plant in the combined compound profile than that produced by the additive alterations in patterns or levels of the first and additional expression profiles.

12. The method according to claim 1, wherein the combination of compounds has an antagonistic effect that is a reduction in the magnitude of the alteration in expression pattern or level of genes or gene products in the combined compound profile than that produced by the pattern or level of expression of the additive alterations of the first and additional profiles.

13. The method according to claim 11, wherein the alteration in the pattern or levels of expression in the combined expression profile is an at least 2-fold alteration in expression of the genes or gene products forming the pattern of the additive alterations of the first and additional profiles.

14. The method according to claim 1, wherein the selected alteration of the gene or gene product expression profile is that gene or gene product expression pattern typical of a plant in a state selected from increased ethylene sensitivity, decreased ethylene sensitivity, enhanced ripening, increased resistance to stress, heat, population density or salinity, decreased resistance to stress, heat, population density or salinity, increased pathogen resistance, decreased pathogen resistance, increased flowering, increased nitrogen efficiency, increased herbicidal resistance, increased photosynthetic activity and increased yield.

15. The method according to claim 1, wherein the additional compound comprises two or more additional compounds.

16. The method according to claim 1, wherein the plant cell is from a plant selected from the group consisting of rice, maize, wheat, barley, sorghum, millet, switchgrass, miscanthus, grass, oats, tomato, potato, banana, kiwi fruit, avocado, melon, mango, cane, sugar beet, tobacco, papaya, peach, strawberry, raspberry, blackberry, blueberry, lettuce, cabbage, cauliflower, onion, broccoli, brussel sprout, cotton, canola, grape, soybean, oil seed rape, asparagus, beans, carrots, cucumbers, eggplant, melons, okra, parsnips, peanuts, peppers, pineapples, squash, sweet potatoes, rye, cantaloupes, peas, pumpkins, sunflowers, spinach, apples, cherries, cranberries, grapefruit, lemons, limes, nectarines, oranges, peaches, pears, tangelos, tangerines, lily, carnation, chrysanthemum, petunia, rose, geranium, violet, gladioli, orchid, lilac, crabapple, sweetgum, maple, poinsettia, locust, ash, poplar, linden tree and Arabidopsis.

17. A method for identifying synergistic compounds that selectively alter wildtype gene expression of a plant cell, the method comprising:
(a) comparing
(i) a first expression profile from a plant cell or tissue contacted with a first test compound;
(ii) an additional expression profile from the same plant cell or tissue contacted with an additional test compound; and
(iii) a combined expression profile from the same plant cell or tissue contacted with the first test compound and the additional test compound,
wherein each plant cell or tissue was contacted for a time sufficient to produce an alteration in the expression of at least one gene or gene product in the plant's cells or tissues; and wherein each expression profile comprises the expression level or pattern of at least one gene or gene product of the plant cell or tissue; and
(b) identifying a significant, unexpected alteration in the level or pattern of expression between the genes or gene products of the combined compound profile (a)(iii) and those of a combination of the first profile (a)(i) and additional profile (a)(ii).

18. The method according to claim 1, wherein the comparing is performed by a computer processor or computer-programmed instrument that generates numerical or graphical data.

19. The method according to claim 1, wherein the first compound is 1-methylcyclopropene.

20. The method according to claim 17, wherein the first test compound is 1-methylcyclopropene.

21. A method for identifying compounds that selectively alter wildtype gene expression of a plant cell, the method comprising:
(a) comparing
(i) a first expression profile from a plant cell or tissue contacted with a first test compound that has unknown effect on the plant cell,
(ii) an additional expression profile from the same plant cell or tissue contacted with an additional test compound that has unknown effect on the plant cell, and
(iii) a combined compound expression profile from the same plant cell or tissue contacted with the first test compound and the additional test compound,
wherein each plant cell or tissue was contacted for a time sufficient to produce an alteration in the expression of at least one gene or gene product in the plant's cells or tissues; and wherein each expression profile comprises the expression level or pattern of at least one gene or gene product of the plant cell or tissue; and
(b) identifying a significant, unexpected alteration in the level or pattern of expression between the genes or gene products of the combined compound profile and those of the additive alterations of the first profile and the additional profile.

22. The method according to claim 21, wherein the plant cell is derived from a plant selected from the group consisting of corn, soybean, wheat, oil seed rape, rice, sugar beets, cotton, oats, barley, sorghum, tobacco and arabidopsis.
